(19) United States
(12) Patent Application Publication
(10) Pub. No.: US 2020/0386766 A1
(43) Pub. Date: Dec. 10, 2020

(54) METABOLIC SIGNATURES ASSOCIATED WITH DIAGNOSIS, DISEASE PROGRESSION, AND IMMUNOLOGICAL RESPONSE TO TREATMENT OF PATIENTS WITH COVID-19

(71) Applicant: Metabolomycs, Inc., Long Beach, CA (US)

(72) Inventors: Robert Nagourney, Long Beach, CA (US); Ismael Silva, Sau Paulo (BR); Paulo D'Amora, San Paulo (BR)

(73) Assignee: Metabolomycs, Inc., Long Beach, CA (US)

(21) Appl. No.: 16/921,113

(22) Filed: Jul. 6, 2020

Related U.S. Application Data
(63) Continuation-in-part of application No. 15/387,932, filed on Dec. 22, 2016, now Pat. No. 10,725,056.
(60) Provisional application No. 63/010,680, filed on Apr. 15, 2020.

(51) Int. Cl. G01N 33/68; G01N 33/569

(52) U.S. Cl. CPC G01N 33/6812; G01N 33/569

(57) ABSTRACT

A system and method for using new biomarkers to assess individual diseases, including, but not limited to, a patient's prognosis before and/or after being diagnosed with the disease. In one embodiment of the present invention, absolute quantification of annotated metabolites by mass spectrometry is used to identify certain biomarkers and derivatives thereof (i.e., signatures), which are then used to screen for, diagnose, predict, prognose, and/or treat various diseases, including, but not limited to, COVID-19.

### BACKGROUND OF THE INVENTION

### 1. Field of the Invention

The present invention relates to metabolic biomarker sets for assessing at least viral diseases. In preferred embodiments, the present invention relates to the use of biomarker sets for screening and/or diagnosing viral infections, for predicting immunologic response of an individual to therapy and/or prognosis of disease progression, and for monitoring of disease activity in the individual. In other embodiments, the invention relates to methods for screening and/or diagnosing viral infections, for prediction of immunologic response of an individual to therapy and/or prognosis of disease progression, and for monitoring of disease activity in the individual, as well as to a kit adapted to carry out the methods. In yet other embodiments, the present invention can be used to screen, diagnose, etc., non-viral diseases in which our inborn immunology is likewise challenged such as bacterial and fungal infections as well as cancer. By employing the specific biomarkers and the methods according to the present invention, it becomes possible to more properly and reliably assess infections (e.g., viral, etc.). In particular, it becomes possible to screen for and diagnose an individual with high accuracy and predict early (e.g., before the individual has been infected, etc.) the individual's response to infection and/or therapy, which may include antivirals, antiretrovirals, antibiotics, etc.

### 2. Description of Related Art

### Viral Infections

There are millions of types of viruses that exist with over 5000 types identified, including swine flu (H1N1), rhinovirus, coronavirus (e.g., COVID-19, SARS, MERS, etc.), Zika virus, Ebola, human papillomavirus (HPV), herpes simplex virus (HSV), and human immunodeficiency virus (HIV), to name a few. Results from being infected can range from mild (e.g., symptoms that are flu-like or resemble the common cold) to severe (e.g., respiratory tract infections, pneumonia, acquired immunodeficiency syndrome (AIDS), etc.), depending on the specific viral infection, therapy, and individual's response thereto.

Viruses contain a small piece of genetic code and are protected by a coat of protein and fat. They invade a host and attach themselves to a cell. As they enter the cell, they release genetic material. The genetic material forces the cell to replicate, and the virus multiplies. When the cell dies, it releases new viruses, and these go on to infect new cells. Not all viruses destroy their host cell. Some of them change the function of the cell. In this way, viruses such as HPV or Epstein-Barr virus (EBV) can lead to cancer by forcing cells to replicate in an uncontrolled way.

Antiviral medications help in some cases. They can either prevent the virus from reproducing or boost the individual's immune system. Other treatments are directed toward relieving symptoms (e.g., fever, shortness of breath, etc.). Clearly, it would be advantageous to understand at the outset how an individual is going to respond to both the infection (itself) and individual therapies, including antiviral and antiretroviral therapy. To this end, the inventors have discovered that this can be accomplished by analyzing an individual's metabolomic profile, including individual metabolites and/or combinations (e.g., ratios) thereof (i.e., metabolomic signatures).

### Metabolomics

Metabolomics is a comprehensive quantitative measurement of low molecular weight compounds covering systematically the key metabolites, which represent the whole range of pathways of intermediary metabolism. The capability to analyze large arrays of metabolites extracts biochemical information reflecting true functional endpoints of overt biological events while other functional genomics technologies such as transcriptomics and proteomics, though highly valuable, merely indicate the potential cause for phenotypic response. Therefore, they cannot necessarily predict drug effects, toxicological response or disease states at the phenotype level unless functional validation is added.

Metabolomics bridges this information gap by depicting in particular such functional information since metabolite differences in biological fluids and tissues provide the closest link to the various phenotypic responses. Needless to say, such changes in the biochemical phenotype are of direct interest to pharmaceutical, biotech and health industries once appropriate technology allows the cost-efficient mining and integration of this information.

In general, phenotype is not necessarily predicted by genotype. The gap between genotype and phenotype is spanned by many biochemical reactions each with individual dependencies to various influences, including drugs, nutrition and environmental factors. In this chain of biomolecules from the genes to phenotype, metabolites are the quantifiable molecules with the closest link to phenotype. Many phenotypic and genotypic states, such as a toxic response to a drug or disease prevalence are predicted by differences in the concentrations of functionally relevant metabolites within biological fluids and tissue.

### HIV/AIDS

By way of example, human immunodeficiency virus infection/acquired immunodeficiency syndrome (HIV/AIDS) is a disease of the human immune system caused by infection with HIV. During the initial infection, a person may experience a brief period of influenza-like illness. This is typically followed by a prolonged period without symptoms. As the illness progresses, it interferes more and more with the immune system, making the person much more likely to get infections, including opportunistic infections and tumors that do not usually affect people who have working immune systems. There is currently no cure or effective HIV vaccine. Treatment consists of antiretroviral therapy (ART), such as high active antiretroviral therapy (HAART) which slows progression of the disease and as of 2010 more than 6.6 million people were taking them in low and middle income countries.

The United States Center for Disease Control and Prevention created a classification system for HIV, and updated it in 2008. This system classifies HIV infections based on CD4 count and clinical symptoms, and describes the infection in three stages, including: Stage 1: CD4 count 500 cells/μl and no AIDS defining conditions; Stage 2: CD4 count 200 to 500 cells/μl and no AIDS defining conditions; and Stage 3: CD4 count 200 cells/μl or AIDS defining conditions. For surveillance purposes, the AIDS diagnosis still stands even if, after treatment, the CD4+T cell count rises to above 200 per μL of blood or other AIDS-defining illnesses are cured. However, it is becoming increasingly evident that the CD4 count and viral load do not provide a complete picture of the underlying state of the immune system for HIV patients. Indeed, the extension of life as a consequence of antiretroviral therapies has heralded a new era of non-AIDS-related diseases and incomplete restoration of immune function despite good control of viral loads. Therefore, the identification and incorporation of new predictive markers for HIV (and other viral) diagnosis and classification is of utmost importance.

In sites where antiretroviral drugs have been widely used since the mid-90s, the use of antiretroviral therapy (ART) has changed the natural course of HIV infection, improving the immune system of patients and thus resulting in both reduced incidence of opportunistic infections and increased survival of HIV-infected patients.

Recent data shows that in Brazil there has been an increase in survival among patients diagnosed with AIDS, with 63.97% of patients achieving a survival of 108 months. Recently, several efforts have been made in order to understand the pathogenesis of HIV by means of the evaluation of its impact on infected cells, on the discovery of disease biomarkers and the understanding of disease progression through the study of specific subgroups of patients.

### COVID-19

The pandemic of the new severe acute respiratory syndrome virus (SARS-CoV-2) has already been confirmed in more than 10,000,000 people on all continents and has been responsible for more than 500,000 deaths worldwide (World Health Organization—official data of Jun. 29, 2020). The only way to treat severe cases is through respiratory support with still unsatisfactory results. To date, there is no efficient pharmacological treatment to modify the natural history of evolution of COVID-19, which results in the mortality of approximately 2% of diagnosed patients, with more than 20% of these progressing with reduced O2 saturation below 94% and pneumonia.

Although the mechanism of action of SARS-CoV-2 is still not completely clear, some groups appear to be more susceptible to the severe form of this infection. Among them are people with pre-existing medical conditions, mainly disorders related to glucose homeostasis (diabetes) and age (hypertension, heart disease, lung disease, asthma, cancer and diabetes) and elderly people. SARS-Cov-2 is known to infect pulmonary epithelial cells and macrophages. Several types of viruses, when infecting target cells, alter their cellular metabolism, inducing pathways favorable to viral replication. This disease is highly contagious, and it is transmitted by inhalation or contact with infected droplets and the incubation period ranges from two to fourteen days.

The symptoms are usually fever, cough, sore throat, breathlessness, fatigue, among others. The disease is mild in most people. While many people are asymptomatic, in some (usually the elderly and those with comorbidities), the virus may progress to pneumonia, acute respiratory distress syndrome (ARDS) and multi organ dysfunction, with a case fatality rate between 2 to 3%. Diagnosis is by demonstration of the virus in respiratory secretions by special molecular tests. Common laboratory findings include normal/low white cell counts with elevated C-reactive protein (CRP). The computerized tomographic chest scan is usually abnormal even in those with no symptoms or mild disease. Treatment is essentially supportive; role of antiviral agents is yet to be established. Prevention entails home isolation of suspected cases and those with mild illnesses and strict infection control measures at hospitals that include contact and droplet precautions.

Clearly, there is a need (urgently) for new screening and diagnosing procedures, which can be easily performed, and which can provide for more accurate and effective results, as well as for a more reliable prediction of a patient's response to ART. In particular, new effective biomarkers for viral (and other disease) screening are urgently needed. To this end, the inventors have analyzed patients (including their metabolomic profiles) with certain viral infections (e.g., HIV/AIDS as a test study) and have extrapolated conclusions and signatures that are applicable to other viral infections (e.g., H1N1, COVID-19, etc.).

These signatures cannot only be used for diagnosis but also prognosis (e.g., for patients that have yet to be infected, patients that are infected but not yet experiencing symptoms, etc.). A universal treatment seems impractical in this moment, as different subsets of patients will likely respond differently. As all drugs have side effects, it will be difficult, but crucial, to tailor treatment regimens based on associated side effects, as well as a patient's disease severity and pre-existing conditions. Thus, the present invention can not only be used to predict how a patient will respond once infected, but also how the patient should be treated. The ability to monitor patient biomarkers across treatment regimens could provide an early assessment of treatment efficacy and side effects, allowing alterations in the course of treatment that prevent adverse outcomes (e.g., predict adverse events stemming from disease progression, side effects of a specific treatment, etc.).

### SUMMARY OF THE INVENTION

Targeted Quantitative MS/MS Analysis

The present invention includes targeted metabolomic analysis of plasma and tissue samples. This validated targeted assay allows for simultaneous detection and quantification of metabolites in plasma and tissue samples in a high-throughput manner. Absolute quantification (μmol/L) of blood metabolites was achieved by targeted quantitative profiling of 186 annotated metabolites by electrospray ionization (ESI) tandem mass spectrometry (MS/MS), blinded to any phenotype information.

Briefly, a targeted profiling scheme was used to quantitatively screen for fully annotated metabolites using multiple reaction monitoring, neutral loss and precursor ion scans. Quantification of metabolite concentrations and quality control assessment was performed in conformance with 21 CFR (Code of Federal Regulations) Part 11, which implies proof of reproducibility within a given error range.

Data Analysis and Validation Tests for metabolomic data analysis, log-transformation was applied to all quantified metabolites to normalize the concentration distributions and uploaded into the web-based analytical pipelines MetaboAnalyst 3.0 (www.metaboanalyst.ca) and Receiver Operating Characteristic Curve Explorer & Tester (ROCCET) available at http://www.roccet.ca/ROCCET for the generation of uni and multivariate Receiver Operating Characteristic (ROC) curves obtained through Support Vector Machine (SVM), Partial Least Squares-Discriminant Analysis (PLS-DA) and Random Forests as well as Logistic Regression Models to calculate Odds Ratios of specific metabolites ROC curves were generated by Monte-Carlo Cross Validation (MCCV) using balanced sub-sampling where two thirds (2/3) of the samples were used to evaluate the feature importance. Significant features were then used to build classification models, which were validated on the 1/3 of the samples that were left out on the first analysis. The same procedure was repeated 10-100 times to calculate the performance and confidence interval of each model. To further validate the statistical significance of each model, ROC calculations included bootstrap 95% confidence intervals for the desired model specificity as well as accuracy after 1000 permutations and false discovery rates (FDR) calculation.

Metabolite Panel

In total, 186 annotated metabolites were quantified using the p180 kit (BIOCRATES Life Sciences AG, Innsbruck, Austria), being 40 acylcanitines (ACs), 21 amino acids (AAs), 19 biogenic amines (BA), sum of hexoses (Hex), 76 phosphatidylcholines (PCs), 14 lysophosphatidylcholines (LPCs) and 15 sphingomyelins (SMs). Glycerophospholipids were further differentiated with respect to the presence of ester (a) and ether (e) bonds in the glycerol moiety, where two letters denote that two glycerol positions are bound to a fatty acid residue (aa=diacyl, ae=acyl-alkyl), while a single letter indicates the presence of a single fatty acid residue (a=acyl or e=alkyl). The participants had their samples additionally analyzed for the following energy metabolism metabolites: lactate, pyruvate/oxaloacetate, alpha ketoglutarate, fumarate and succinate.

Metabolites and Ratios for Viral Infections

A descriptive analysis of 28 blood metabolites, including their correlation with immune response to viral infection, is shown in Table 1 (below). Very low concentrations of sphingomyelins and dopamine in parallel with high levels of dicarboxylicacylcarnitines, L-aspartate and many plasmalogen/plasminogen phosphatidylcholines, such as PC ae C38:1 and PC ae C40:3, were detected in the blood of patients with viral infection compared with controls.

TABLE 1
The top 28 metabolites whose concentrations were statistically
elevated or decreased in virally infected patients compared to controls.

| Metabolite | Correlation | T test | pValue | FDR |
|---|---|---|---|---|
| PC ae C38:1 | 0.84706 | 10.205 | 8.0647E−13 | 1.9306E−5 |
| C5-M-DC | 0.84089 | 99.488 | 1.71E−08 | 2.01E−07 |
| lysoPC a C24:0 | 0.82279 | 92.698 | 1.29E−07 | 1.44E−06 |
| C5:1-DC | 0.79126 | 82.856 | 2.69E−06 | 2.68E−05 |
| Glutamate | 0.71738 | 65.935 | 6.20E−04 | 5.58E−03 |
| PC ae C40:3 | 0.57136 | 4.4578 | 6.2893E−5 | 1.082E−11 |
| Aspartate | 0.7492 | 72.427 | 7.50E−05 | 7.09E−04 |
| PC aa C42:5 | 0.6687 | 57.588 | 9.53E−03 | 8.19E−02 |
| SM C26:0 | −0.57777 | −4.5326 | 4.9721E−05 | 3.0639E−4 |
| lysoPC a C14:0 | −0.63814 | −53.072 | 4.15E−02 | 3.14E−02 |
| PC aa C30:0 | −0.64955 | −54.703 | 2.44E−02 | 1.92E−01 |
| PC aa C28:1 | −0.66539 | −57.075 | 1.13E−03 | 9.26E−02 |
| SM C26:1 | −0.79524 | −83.987 | 1.89E−06 | 1.99E−06 |
| C12-DC | −0.8409 | −99.493 | 1.70E−08 | 2.01E−07 |
| SM C20:2 | −0.8444 | −10.093 | 1.12E−08 | 1.51E−11 |
| Nitrotyrosine | −0.86159 | −10.869 | 1.20E−10 | 1.75E−08 |
| Dopamine | −0.86968 | −11.282 | 3.79E−11 | 5.97E−10 |
| SM C18:1 | −0.87877 | −11.791 | 9.42E−11 | 1.62E−09 |
| SM C18:0 | −0.88526 | −12.187 | 3.26E−12 | 6.15E−10 |
| SM (OH) C16:1 | −0.89156 | −12.605 | 1.09E−11 | 2.28E−10 |
| SM C16:0 | −0.90533 | −13.649 | 7.69E−13 | 1.82E−11 |
| SM (OH) C24:1 | −0.91078 | −14.124 | 2.40E−13 | 6.49E−12 |
| SM (OH) C14:1 | −0.91275 | −14.307 | 1.55E−13 | 4.88E−12 |
| SM C24:1 | −0.91696 | −14.716 | 5.85E−14 | 2.21E−12 |
| SM C16:1 | −0.92619 | −15.729 | 5.71E−15 | 2.70E−13 |
| SM (OH) C22:1 | −0.93876 | −17.445 | 1.40E−16 | 8.83E−15 |
| SM (OH) C22:2 | −0.94566 | −18.622 | 1.29E−17 | 1.22E−15 |
| SM C24:0 | −0.94912 | −19.298 | 3.47E−19 | 6.56E−16 |

FDR = False Discovery Rate;
C5-M-DC = Methylglutarylcarnitine;
lysoPC a C24:0 = Glycerophospholipids;
C5:1-DC = Glutaconylcarnitine;
PC aa C42:5 = Glycerophospholipids;
lysoPC a C14:0 = Glycerophospholipids;
PC aa C30:0 = Glycerophospholipids;
Phosphatidylcholines PC aa C28:1 = Glycerophospholipids;
C12- DC = Dodecanedioylcarnitine;
SM = Sphingomyelin.

The severe deregulation in acylcarnitine and sphingomyelin metabolism suggests that viral infection with RNA viruses like HIV, COVID-19 and others, leads to deficiencies in mitochondrial function. Therefore, the inventors assembled ratios of certain metabolite concentrations as proxies for enzymatic activity. They then examined the proportion of esterified to free carnitines, β- and O-oxidation, and the rate-limiting step in the uptake of fatty acids into the mitochondria related to carnitine palmitoyl transferase I (CPT1) activity. They also examined the SYNE2 locus because of its relation to SGPP1 (sphingosine-1-phosphate phosphatase 1) activity, a key player in the sphingosine rheostat that governs the interchange between pro-apoptotic ceramides and S1P, a well-established ligand in survival signaling.

ANOVA statistical analysis confirmed the hypothesis by demonstrating that viral infection is associated with a substantial deterioration in mitochondrial function. This conclusion is supported by a decrease in the proportion between esterified and free carnitines ((Total esterified carnitines(AC)/free carnitines (CO)) (p=9.8245E-11 and False Discovery Rate (FDR)=4.1977-10), decreased β-oxidation (p=1.3529E-13 and FDR=8.4782E-13) in parallel with increased O-oxidation (p=6.9445E-11 and FDR=3.1085E-10), and decreased uptake of fatty acids by the mitochondria (CPT1) (p=0.0016126 and FDR=0.0026136). As a consequence, the direct products of normal mitochondria, such as non-essential amino acids (p=1.5306E-47 and FDR=7.1938E-46) and sphingomyelins (p=1.1088E-18 and FDR=6.74E-19) were down-regulated in patients with viral infection.

Disturbances in fatty acid oxidation (FAO), as revealed by declines in CPT1 and (3-oxidation functions, were recently reported to be very important in T cell survival and the promotion of CD8+ TM cell development. Furthermore, it has been shown that perturbations on sphingolipids and glycerophospholipids altering membrane lipid composition may impair innate immune responses. β-oxidation is particularly down-regulated (p=2.5195E-8; FDR=1.1412E-7) among INR.

Furthermore, there was a significant decline in sphingosine-1-phosphate phosphatase 1 activity (SGPP1, SYNE2 locus) after treatment, particularly among INR, when evaluated by the ratio PC aa C28:1/PC ae C40:2 (p=8.4667E-7, -log10(p)=6.0723, FDR=1.2712E-5).

Importantly, Sphingosine-1-Phosphate (S1 P) is involved in lymphocyte egress from lymphoid organs and bone marrow into circulatory fluids via a gradient of S1 P. Because SGPP1 (SYNE2 Locus) is correlated to CD4+T cell counts (p=0.0071195; FDR=0.16446), it is tempting to speculate the existence of a link between Sphingosine-1-Phosphate Phosphatase 1 activity and INR.

The amount of ether lipids as measured by the total acyl-alkyl-containing phosphatidylcholines to total phosphatidylcholines (AGPS) ratio was down-regulated after 1 year of follow-up in all groups but INR (p=1.1405E-5, -log10(p)=4.9429, FDR=9.6586E-5). Because ether lipids activate thymic and peripheral semi-invariant natural killer T cells known to be evolutionarily conserved lipid reactive T cells, it was hypothesized that the metabolic enzyme alkylglycerone phosphate synthase (AGPS), a critical step in the synthesis of ether lipids, could be aberrantly activated among INR, leading to impaired CD4+ T cell recovery. The inventors have evaluated ether lipid biosynthesis activity using HIV infected patients as a model for viral infection response and examined their results after they received anti-retroviral treatment vis a vis viral load level and CD4/CD8 in all patients who naturally control viremia (Elite controllers) or Immunological Responders.

Using the results in this HIV patient population they found a significant negative correlation (p=8.5025E-7; FDR=1.1053E-4) between Ether Lipids (AGPS) and increasing levels of CD4 (from 160 to 1215 mm3) (PostHoc=160 >1215; 361 >1215), with opposite results observed for increases in viral load (p=8.5025E-7 Log10(p)=4.9429, FDR=1.1053E-4). In addition, the amount of ether lipids remains elevated among INR even during periods of undetectable viral load (p=1.1537E-4, FDR=3.5435E-4) when significant declines in SGPP1 (p=1.0626E-20, FDR=3.046E-19) and in β-Oxidation (p=3.3247E-5,FDR=1.0212E-4) are also observed. Lipid alterations in these infected individuals receiving protease inhibitor-based antiretroviral treatment determined using untargeted metabolomic profiling of plasma, has been previously linked to markers of inflammation, microbial translocation, and hepatic function, suggesting that dysregulated innate immune activation and hepatic dysfunction are occurring among virally infected patients as demonstrated in these HIV antiretrovirally-treated individuals.

Furthermore, metabolomic profile in infected children shows hypoleptinemia and hypoadiponectinemia and is the activation of critical adipose tissue storage and function in the adaptation to malnutrition. Also, alterations in the Cerebrospinal fluid metabolome among antiretrovirally-treated individuals harboring viral-associated neuro-cognitive disorders reveal that persistent inflammation, glial responses, glutamate neurotoxicity, and altered brain waste disposal are associated with cognitive alteration. As cognitive dysfunction has been described in patients with viral infections, the observed changes are consistent with a growing recognition of viral impact on neurological tissues.

During the study, the presence of a metabolomic signature that can be used to identify “Rapid Progression” and “INR” at baseline was investigated. A combination of five different metabolites and ratios were able to accurately identify Rapid Progressors or INR at baseline with 88.89% sensitivity, 92.31% specificity, 88.89% positive predictive value and 92.31% negative predictive value (AUC=0.871; 95% CI: 0.619-1; p=0.01). During the discovery phase, the results repeatedly pointed to metabolites and ratios linked to metabolism affecting acylcarnitine hydroxylation and carboxylation as well as the catabolism of branched chain amino acids, lysine, organic acids, and tryptophan (see Table 1 above).

Notably, when elevated, as seen among viral Elite controllers, these biochemical markers are highly suggestive of an inborn error of metabolism named late-onset multiple acyl-coenzyme A dehydrogenase deficiency (MADD, MIM#231680). Therefore, we quantified the amount of organic acids, branched chain amino acids and lysine as a diagnostic approach for MADD, in addition to using the ratio C7-DC/C8 as a proxy to analyze the activity of a MADD related enzyme, electron-transferring flavoprotein dehydrogenase (ETFDH). The results demonstrated increased levels of alpha aminoadipic acid (p=0.029658, -log10(p)=1.5279, FDR=0.078855), lysine (p=0.02768, -log10(p)=1.5578, FDR=0.075369) and Branch Chain Amino Acids (BCAA) (p=3.2721E-12,-log10(p)=11.485, FDR=1.6189E-11) among Elite controllers.

Moreover, the ETFDH activity is significantly less active among Elite controllers compared to the other infected groups (T-Test=6.505E-4) and to uninfected controls (T-Test=0.0092744). Therefore, possibly an inborn error of metabolism (MADD) and its reduction of ETFDH activity, which can be asymptomatic in many individuals, relates to a control of viral infection and viral replication and a functional cure of viral infections, including HIV, Coronavirus, etc.

The results presented here make it clear that in addition to the invention's utility as reliable biomarkers, metabolomic profiles of viral infected individuals can provide insights into mechanisms of virus related tissue and organ damage, and further the development of interventional strategies, such as fixing the decrease levels of dopamine seen among the infected subjects in this study. Of note, low dopamine levels have been implicated in the mechanisms of psychiatric diseases such as depression and schizophrenia. As an example and corroborating the predicative abilities of the metabolic signatures identified in blood collected at baseline, of patients that years later developed specific viral phenotypes, a recent study have been able to identify functional annotations that accurately predicted the inflammatory response of cells derived from patients suffering from inborn errors of metabolism solely on their altered membrane lipid composition.

It should be appreciated that the present invention is not limited to the foregoing results, and further results, including equations and/or ratios that are important in assessing viral infections (e.g., COVID-19) are provided below, in the Detailed Description section. Also provided below are detailed discussions on how the present invention can be used to predict how a patient will respond once infected (e.g., respond to the infection, respond to therapy, etc.), and examples of conditions associated with different immunological responses with respect thereto.

Determining and Providing Results

The invention may involve a patient visiting a doctor, clinician, technician, nurse, etc., where blood or a different sample is collected. The sample would then be provided to a laboratory for analysis, as discussed above (e.g., mass spectrometry, log-transformation, comparisons, etc.). In another embodiment, a kit can be used to obtain the sample, where the kit is made available to the patient via a medical facility, a drug store, the Internet, etc. In this embodiment, the kit may include one or more wells and one or more inserts impregnated with at least one internal standard. The kit can be used to gather the sample from a patient, where the sample is then provided to a laboratory for analysis.

It should be appreciated that the analysis is preferably performed via software, where initial results (data post mass spectrometry, post log-transformation), are stored in memory, presented on a display (e.g., computer monitor, etc.) and/or printed. The initial results can then be compared to known “signatures” for different viral infections, where similarities and differences are used to screen for, diagnose, prognose, treat, etc. a particular virus. It should be appreciated that the sample may be assessed for a particular virus, or for multiple viruses, depending on the patient's sex, age, etc. Thus, the software could be used to assess a particular virus or assess at least one virus from a plurality of viruses.

It should also be appreciated that the “comparing” step can be performed by (i) software, (ii) a human, or (iii) both. For example, with respect to the prior, a computer program could be used to compare sample results to known signatures and to use differences and/or similarities thereof to assess at least one viral infection, and provide diagnosis, prognosis, and/or treatment for the same. Alternatively, in the second embodiment, a technician could be used to compare sample results to known signatures (or aspects thereof) and make a diagnosis, prognosis, and/or treatment decision based on perceived similarities and/or differences. Finally, with respect to the latter, a computer program could be used to plot (e.g., on a computer display) sample results alongside known signatures (e.g., signatures of healthy patients, signatures of unhealthy patients, life expectancies, etc.). A technician could then view the same and make at least one diagnosis, prognosis, treatment recommendation, etc. based on similarities and/or differences in the plotted information.

Bottom line, it is the differences and/or similarities between known signatures that allows a virus (including treatments and/or therapies therefore) to be assessed, whether that assessment is automated (e.g., performed by a computer), performed manually (e.g., done by a human), or a combination of the two. Results (e.g., assessments) are then provided to the patient directly (e.g., via mail, an electronic communication, etc.) or via the patient's doctor, and can include screening information, diagnosis information, prognosis information, and treatment information.

For example, the invention can be used to distinguish a sample that is COVID-19 positive from one that is not. If it is positive, then the invention can further be used to define the virus (e.g., by degree, treatability, etc.). This can be done using terminology (e.g., mild, severe, lethal, etc.), at least one scale (e.g., 1-10, 1-100, A-F, etc.), where one end of the scale is low grade (e.g., mild) and the other end is high grade (e.g., lethal), or other visual forms (e.g., color coded, 2D or 3D model, etc.).

The invention can also be used to provide a prognosis. For example, the invention can be used to provide gradations within the signature (or signatures), subcategorizing the patient into one that is likely to survive, likely to be asymptomatic, likely to result in pneumonia, likely to require ventilation, etc. Again, prognosis could be provided using terminology (e.g., low risk, medium risk, high risk, etc.), at least one scale, or other visual forms.

The invention can also be used to screen for viruses. Medical screening is the systematic application of a test or inquiry to identify individuals at sufficient risk of a specific disorder to benefit from further investigation or direct preventative action (these individuals not having sought medical attention on account of symptoms of that disorder). The present invention uses metabolic signatures to screen for viral infections in populations who are considered at risk. For most viral infections, like COVID-19, this may be older individuals with underlying medical conditions (e.g., asthma, bronchitis, etc.).

It should be appreciated that while several examples have been provided as to what the present invention can discern from a blood sample (or the like), the present invention is not so limited, and other types of diagnosis and prognosis, including treatments, are within the spirit and scope of the present invention. Once a sample has been received and processed (e.g., processed using techniques like the one used to identify the signatures in the first place, such as mass spectrometry (to quantify metabolites), log-transformation (or other mathematical manipulation to normalize the data), etc.), the initial results (e.g., metabolites and/or sets thereof) can then be compared to signatures (or portions thereof) that have been identified (by the inventors) as useful in assessing at least one virus and/or therapeutic response thereto.

The signatures may be stored in memory, and the initial data (i.e., processed sample) may be compared to at least one signature either manually (e.g., by viewing the sample, or initial results thereof, against known signatures), automatically (e.g., using a computer program to discern differences and/or similarities between the sample, or initial results thereof, and known signatures), or both (e.g., a program determines at least one diagnosis/prognosis and a technician reviews the data to validate the same). Based on the results (i.e., comparison results), at least one diagnosis and/or prognosis, which may or may not include treatment, is identified and provided to the patient.

### Conclusion

Having thus described several embodiments of a system and method for using new biomarkers for assessing different viral infections, it should be apparent to those skilled in the art that certain advantages of the system and method have been achieved. It should also be appreciated that various modifications, adaptations, and alternative embodiments thereof may be made within the scope and spirit of the present invention.

For example, it should be appreciated that while a first viral infection (e.g., HIV) may have a first signature, and a second viral infection (e.g., COVID-19) may have a second, different signature, the method used in identifying each signature is very similar, and in certain instances identical. Thus, while different viruses have been discussed, for the sake of brevity, details concerning how a signature is identified and subsequently used to assess a particular virus are equally applicable to other signatures and other viruses unless stated otherwise.

It should also be appreciated that a viral infection may have more than one signature or portions thereof. For example, a first signature may be used for diagnoses, a second signature (or portion of the first signature) may be used for prognoses, etc. It should further be appreciated that while a viral infection may have more than one signature, there may be individual aspects (e.g., individual metabolites or derivatives thereof) that are common to several signatures, and can therefore provide, in and of themselves, information on diagnosis, prognosis, treatment, etc.

It should also be appreciated that the present invention is not limited to any particular virus. Those skilled in the art will understand that the methods disclosed herein can be used to identify signatures for, and assess, other viral infections, including those not specifically mentioned herein. The present invention can also be used to identify signatures for, and assess, non-viral infections, such as bacterial infections, fungal infections, etc.

As response to infectious agents draws upon human immunity both innate and adaptive, each individual's immune response reflects their underlying physical wellbeing. Metabolic signatures have the capacity to measure each individual's metabolic health using metabolites and metabolite ratios as metrics. These identify the responsiveness and robustness of each individual's immune system. Thus, the foregoing methods can be used to identify signatures that can also, or alternatively, diagnose, prognose, etc., bacterial infections including staphylococcus, streptococcus, Escherichia coli, Klebsiella, Psuedomonas, to name a few, as well as fungal infections including, but not limited to, Candida, Fusarium, Aspergillus, Coccidioidomycosis, Histoplasmosis, Crytpcoccus, and parasitic infections including Amoeba, Babesiosis, Trypanosomes, Leishmaniasis, Plasmodia and others.

Finally, the present invention is not limited to use of mass spectrometry, or any particular type of mass spectrometry (e.g., electrospray ionization (ESI) tandom mass spectrometry (MS/MS), etc.), and includes other methods for quantifying metabolites, such as chromatography or spectrometry. That being said, the inventors have found that there are benefits to using mass spectrometry, and in particular ESI MS/MS, and the data analysis described herein (e.g., Unsupervised and Supervised Uni and Multivariate Statistics and Machine Learning Procedures). As such, the methods described in detail herein are preferred embodiments, and should be treated as such.

A more complete understanding of a system and method for using metabolic biomarker sets for screening and/or diagnosing viral infections (e.g., COVID-19, etc.), for predicting immunologic response of an individual to therapy and/or prognosis of disease progression, and for monitoring of disease activity in the individual, will be afforded to those skilled in the art, as well as a realization of additional advantages and objects thereof, by a consideration of the following detailed description of the preferred embodiment. Reference will be made to the appended sheets of drawings, which will first be described briefly.

### BRIEF DESCRIPTION OF THE DRAWINGS

FIG. 1 illustrates a method in accordance with one embodiment of the present invention as to how a metabolic signature for a disease is identified and subsequently used to assess a patient's blood sample as to that disease;

FIGS. 2-6 provide a list of analytes, including their abbreviations, that are considered metabolites (or sets thereof) used in certain embodiments of the present invention;

FIGS. 7A and B provide a list of ratios that have been identified as useful in assessing different types of diseases;

FIG. 8 provides a list of parameters that have been identified as useful in assessing certain diseases;

FIG. 9 provides a list of additional ratios that have been identified as useful in assessing certain diseases;

FIG. 10 provides likelihood ratios, and interpretations thereof, used by the inventors during performance of Statistical Univariate Analysis;

FIGS. 11, 12A, and 12B show certain metabolites and ratios that are useful in assessing a patient for HIV, including diagnosis and prognosis related thereto, and have since been found to be useful in assessing patients for other viruses (e.g., COVID-19, etc.);

FIG. 13 provides equations that are useful in assessing a patient for COVID-19, including, but not limited to, determining a prognosis for a patient that may contract COVID-19 (e.g., their immunological response);

FIGS. 14A-H illustrate certain ratios that are useful in assessing a patient for COVID-19, including, but not limited to, determining a prognosis for a patient that may contract the same; and

FIGS. 15A-C illustrate additional metabolites and ratios that have been found to be useful in assessing a patient for COVID-19, including a prognosis for the patient if the patient were to contract the same.

### DETAILED DESCRIPTION OF THE PREFERRED EMBODIMENT

Preferred embodiments of the present invention involve use of targeted metabolomics to assess at least viral diseases, such as COVID-19, or more particularly, to screen and/or diagnose viral infections, predict immunological response of an individual to viral infections, therapies, and/or prognosis of disease progression, and monitor for disease activity. In other embodiments, the invention relates to methods for screening and/or diagnosing viral infections, for prediction of immunologic response of an individual to therapy and/or prognosis of disease progression, and for monitoring of disease activity in the individual, as well as to a kit adapted to carry out the methods. In yet other embodiments, the present invention can be used to screen, diagnose, etc., non-viral diseases such as bacterial infections, fungal infections, etc.

By employing the specific biomarkers and the method according to the present invention it becomes possible to more properly and reliably assess infections (e.g., viral, etc.). In particular, it becomes possible to screen for and diagnose an individual with high accuracy and predict early in advance the individual's response to a virus (e.g., COVID-19) and/or therapy associated therewith, which may include antivirals, antiretrovirals, antibiotics, etc.

It should be appreciated that while a first virus (e.g., HIV) may have a first signature, and a second disease (e.g., COVID-19) may have a second, different signature (e.g., having portions that are similar and portions that are different), the method used in identifying each signature is very similar, and in certain instances identical. Thus, while different viruses have been discussed in different sections below, for the sake of brevity, details concerning how a signature is identified and subsequently used to assess a particular virus are equally applicable to other signatures and other viruses and/or diseases unless stated otherwise. For example, details concerning absolute quantification of annotated metabolites by mass spectrometry provided in the HIV section applies equally to the COVID-19 section, as do other details, unless stated otherwise.

It should also be appreciated that a virus or disease may have more than one signature or portions thereof. For example, a first signature may be used for diagnoses, a second signature (or portion of the first signature) may be used for prognoses, etc. It should also be appreciated that while a virus or disease may have more than one signature, there may be individual aspects (e.g., individual metabolites or derivatives thereof) that are common to several signatures, and can therefore provide, in and of themselves, information on diagnosis, prognosis, treatment, etc. Specifics concerning signatures will be discussed in the corresponding sections below.

It should further be appreciated that the present invention is not limited to any particular virus or disease, and that those skilled in the art will understand that the methods disclosed herein can be used to identify signatures for, and assess, other diseases, including those not specifically mentioned herein. The present invention is also not limited to use of mass spectrometry, or any particular type of mass spectrometry (e.g., electrospray ionization (ESI) tandom mass spectrometry (MS/MS), etc.), and includes other methods for quantifying metabolites, such as chromatography or spectrometry. That being said, the inventors have found that there are benefits to using mass spectrometry, and in particular ESI MS/MS, and the data analysis described herein (e.g., log-transformation, ROC curves, etc.). As such, the methods described in detail herein are preferred embodiments, and should be treated as such.

Prior to discussing the inventions, including individual signatures, the methods used to identify the same, and assess various diseases, certain definitions of term or phrases used herein will first be provided.

Definitions

By employing the biomarkers (or specific sets thereof) and the methods according to the present invention it has become possible to assess a disease (e.g., HIV, COVID-19, etc.) with improved accuracy and reliability. It has surprisingly been achieved in the present invention to provide biomarkers or biomarker sets by measuring the amount and/or ratios of certain metabolites in samples, such as blood samples, of subjects that make it possible to screen, diagnose, and prognose diseases (e.g., COVID-19, etc.) in an improved manner and at early stages.

In general, a biomarker is a valuable tool due to the possibility to distinguish two or more biological states from one another, working as an indicator of a normal biological process, a pathogenic process or as a reaction to a pharmaceutical intervention.

A metabolite is a low molecular compound (<1kDa), smaller than most proteins, DNA and other macromolecules. Small changes in activity of proteins result in big changes in the biochemical reactions and their metabolites (=metabolic biomarker, looking at the body's metabolism), whose concentrations, fluxes and transport mechanisms are sensitive to diseases and drug intervention.

This enables getting an individual profile of physiological and pathophysiological substances, reflecting both genetics and environmental factors like nutrition, physical activity, gut microbial and medication. Thus, a metabolic biomarker gives more comprehensive information than for example a protein or hormone, which are biomarkers, but not metabolic biomarkers.

In view thereof, the term “metabolic biomarker” or short “biomarker” as used herein is defined to be a compound suitable as an indicator of the presence and state of a disease (e.g., COVID-19), including a corresponding prognosis, being a metabolite or metabolic compound occurring during metabolic processes in the mammalian body.

The terms “biomarker” and “metabolic biomarker” are in general used synonymously in the context of the present invention and typically refer to the amount of a metabolite or to the ratio of two or more metabolites. Hence, the term metabolic biomarker or biomarker is intended to also comprise ratios (or other mathematical relationships) between two or more metabolites.

The term “amount” typically refers to the concentration of a metabolite in a sample, such as blood sample, and is usually given in micromol/L, but may also be measured in other units typically used in the art, such as g/L, mg/dL, etc. The term “sum” typically means the sum of molar amount of all metabolites as specified in the respective embodiment.

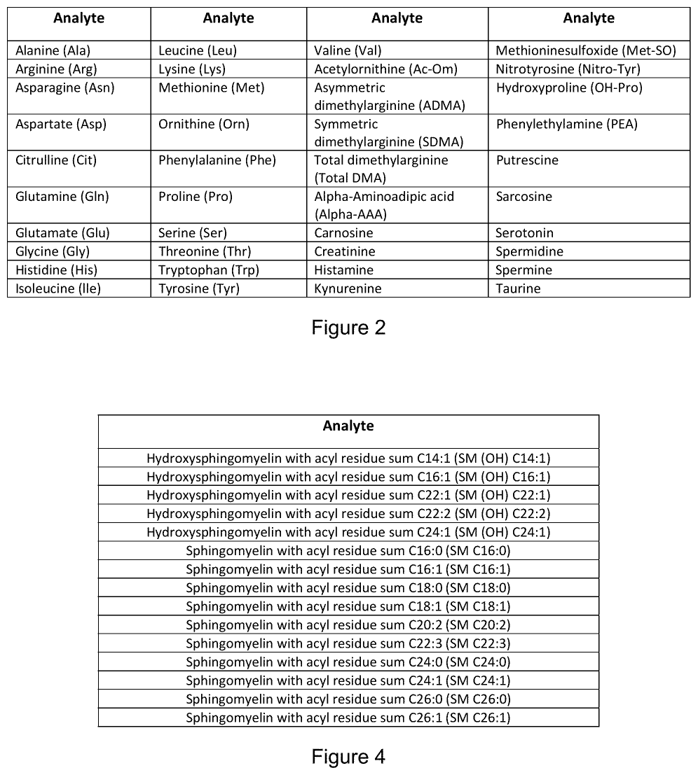

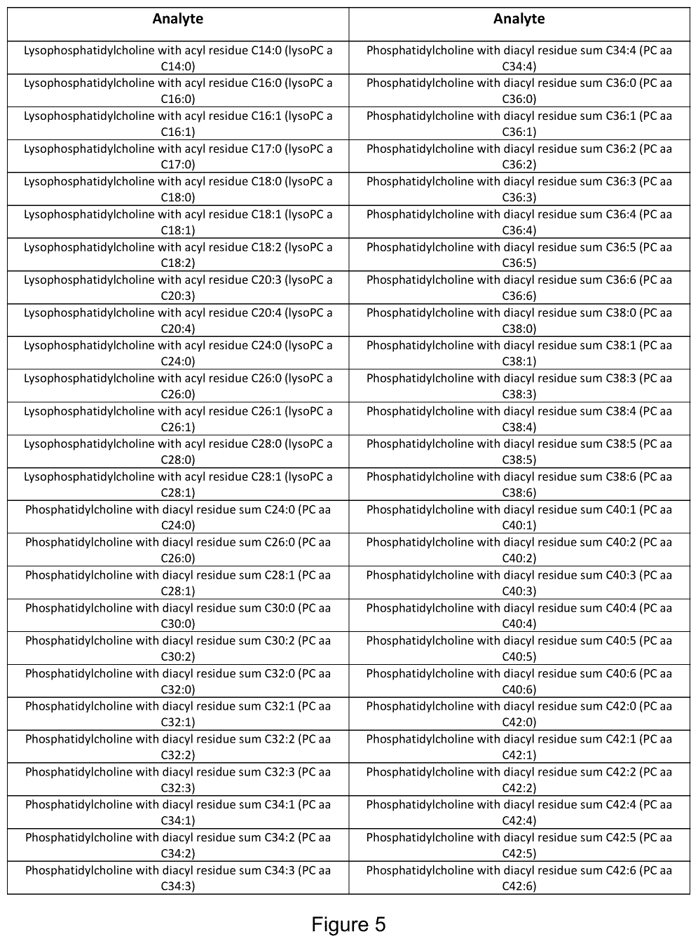

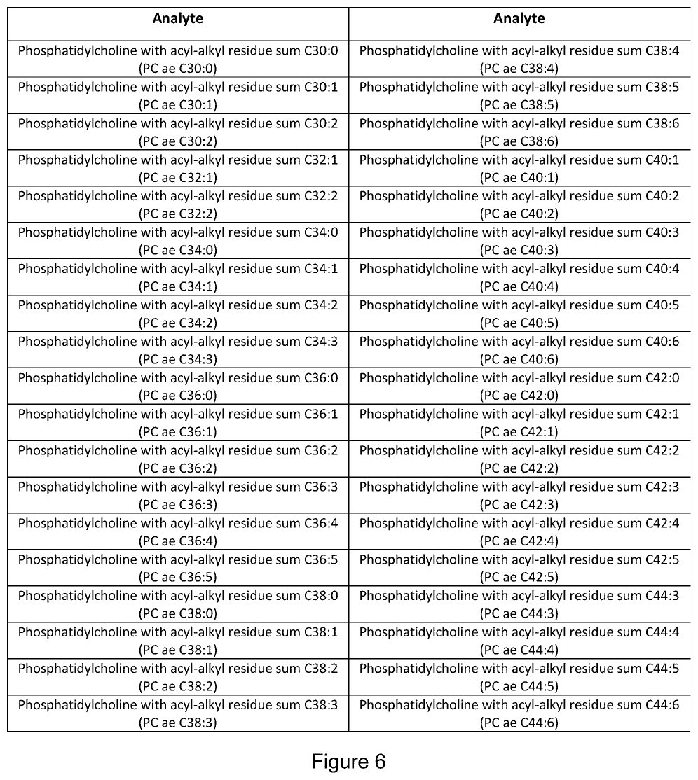

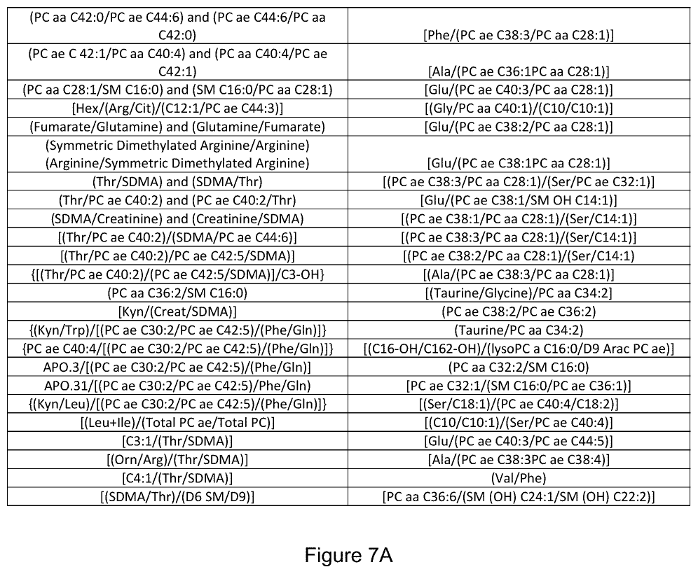

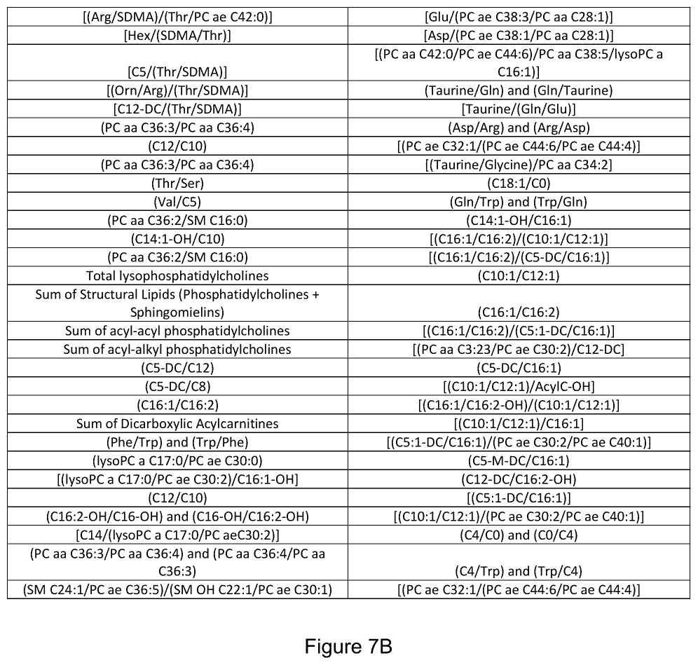

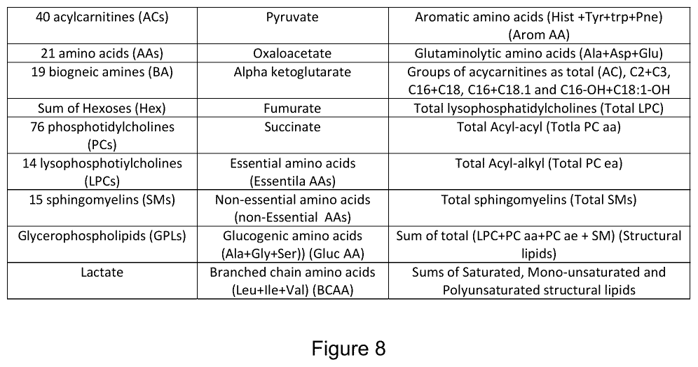

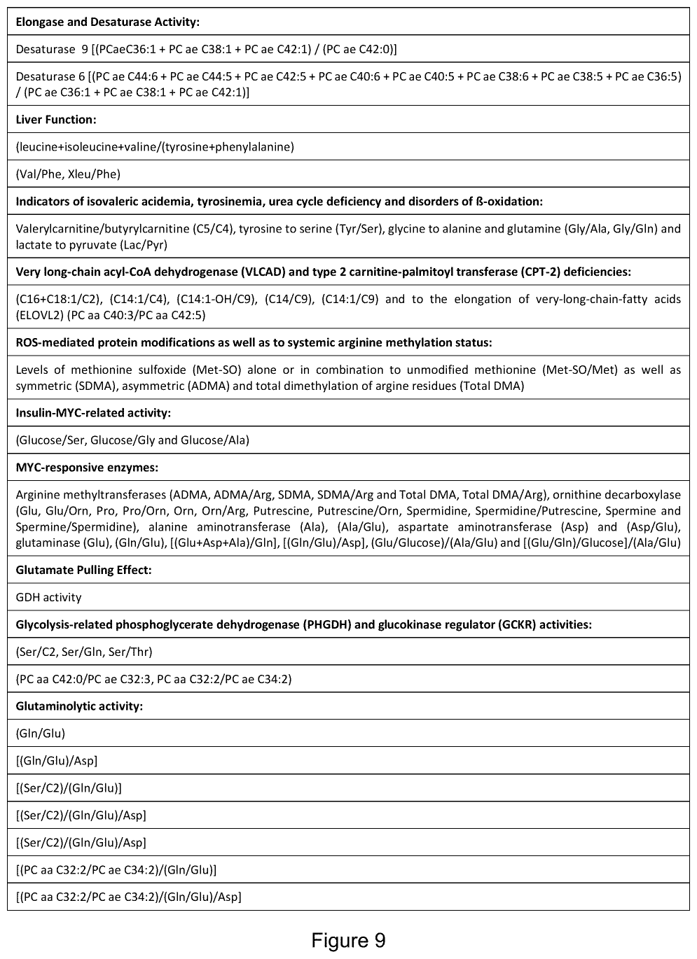

The term “ratio” typically means the ratio of amounts of metabolites as specified in the respective embodiment. The metabolic biomarker (set) measured according to the present invention may comprise the classes of metabolites (i.e. analytes) of amino acids and biogenic amines, acylcarnitines, hexoses, sphingolipids and glycerophospholipids, as listed in FIGS. 2-6.

Biogenic amines in FIG. 2 are understood as a group of naturally occurring biologically active compounds derived by enzymatic decarboxylation of the natural amino acids. A biogenic substance is a substance provided by life processes, and the biogenic amines contain an amine group.

It has surprisingly been found that measuring a set of biomarkers comprising these classes of metabolites, i.e., measuring the amount and/or ratios of certain indicative metabolites, allows for screening and diagnosing various diseases (e.g., HIV, etc.) in an improved manner and at an early stage and allows for assessing biochemical reflection of disease activity, allowing for the prediction of a therapeutic response as well as for sub classification among a disease's behavior.

While a modified “signature” can be used, if one metabolite or one class of metabolites as specified for the respective biomarker combination is omitted or if the number thereof is decreased, the assessment of the disease becomes less sensitive and less reliable.

This particularly applies for the early stages of the disease being not reliably detectable according to known methods using known biomarkers at all. In fact, the measurement of the metabolites contained in the respective sets of biomarkers at the same time allows a more accurate and more reliable assessment of a disease, typically with (A) a sensitivity of greater than 80%, preferably greater than 90%, and more preferably greater than 98%, (B) a specificity of greater than 80%, preferably greater than 85%, and more preferably greater than 90%, (C) a positive predictive value (PPV) of greater than 40%, preferably greater than 50%, and more preferably greater than 60%, and (D) a negative predictive value (NPV) of greater than 80%, preferably greater than 90%, and more preferably greater than 98%. Obviously, biomarkers (or sets thereof) that can reach or achieve 100% (or near 100%) sensitivity, specificity, PPV, and/or NPV is desired.

The meanings of the terms “sensitivity”, “specificity”, “positive predictive value” and “negative predictive value” are typically known in the art and are defined in the context of the present invention according to the “Predictive Value Theory”, as established by the University of Iowa, USA. In this theory, the diagnostic value of a procedure is defined by its sensitivity, specificity, predictive value and efficiency. Description of the formulae are summarized below.

Sensitivity of a test is the percentage of all patients with disease present who have a positive test. (TP/(TP+FN)) x 100=Sensitivity (%) where TP=Test Positive; FN=False Negative.

Specificity of a test is the percentage of all patients without disease who have a negative test. (TN/(FP+TN))×100=Specificity (%) where TN=Test Negative; FP=False Positive.

Predictive value of a test is a measure (%) of the times that the value (positive or negative) is the true value, i.e. the percent of all positive tests that are true positives is the Positive Predictive Value ((TP/(TP+FP))'100=Predictive Value of a Positive Result (%); ((TN/(FN+TN))×100=Predictive Value Negative Result (%))

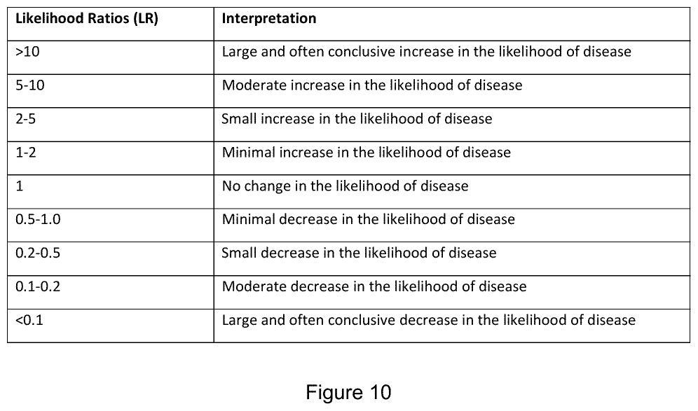

Likelihood Ratios: The performance of biomarkers can further be assessed by determining the Positive and Negative Likelihood Ratios (LR) used herein during Statistical Univariate Analysis (see FIG. 10).

Multivariate Data Analysis: Training cases were used for marker discovery and to identify any clinical variable that might be associated with a disease by logistic regression analysis. Quantification of metabolite concentrations and quality control assessment was performed with software. Internal standards serve as the reference for the metabolite concentration calculations. An xls file was then exported, which contained sample names, metabolite names and metabolite concentration with the unit of μmol/L of in plasma.

Data was then uploaded into the web-based analytical pipeline MetaboAnalyst 2.0 (www.metaboanalyst.ca) and normalized using MetaboAnalyst's normalization protocols (Xia et al 2012) for uni and multivariate analysis, high dimensional feature selection, clustering and supervised classification, functional enrichment as well as metabolic pathway analysis.

Data was also imported to ROCCET (ROC Curve Explorer & Tester) available at http://www.roccet.ca/ROCCET/for the generation of uni and multivariate Receiver Operating Characteristic (ROC) curves obtained through Support Vector Machine (SVM), Partial Least Squares-Discriminant Analysis (PLS-DA) and Random Forests.

Curves were generated by Monte-Carlo cross validation (MCCV) using balanced subsampling where two thirds (2/3) of the samples were used to evaluate the feature importance. Significant features were then used to build classification models, which were validated on the 1/3 of the samples that were left out. The same procedure was repeated multiple times to calculate the performance and confidence interval of each model.

Up and down regulation: An up-regulation means an increase in the concentration of a metabolite, e.g., an increase in the rate of at which this biochemical reaction occurs due to for example a change in enzymatic activity. For a down-regulation, it's the other way around.

T-test: The t-test is a statistical hypothesis test and the one used is the one integrated in the MarkerView software and is applied to every variable in the table and determines if the mean for each group is significantly different given the standard deviation and the number of samples, e.g., to find out if there is a real difference between the means (averages) of two different groups.

P-value: The p-value is the probability of obtaining a result at least as extreme as the one that was actually observed, assuming that the null hypothesis (the hypothesis of no change or effect) is true. The p-value is always positive and the smaller the value the lower the probability that it is a change occurrence. A p-value of 0.05 or less rejects the null hypothesis at the 5% level, which means that only 5% of the time the change is a chance occurrence. This is the level set in the tables provided herein.

Log-fold change: Log-fold change is defined as the difference between the average log transformed concentrations in each condition. This is a way of describing how much higher or lower the value is in one group compared to another. For example, a log-fold change of 0.3 is “equivalent” to an exp (0.3)=1.34 fold change increase compared to the control (healthier group). Further, a log-fold change of −0.3 is “equivalent” to a exp(−0.3)=0.74=(1/1.34) fold change increase compared to the control or decrease fold change of 1.34 to the disease.

Signatures for particular diseases, including the identification thereof and use of the same for assessing (e.g., screening, diagnosing, prognosing, treating, etc.) particular diseases, will now be discussed.

HIV—Patients, Methodology, and Signature

Initially, the inventors evaluated plasma samples HIV-infected individuals with different phenotypic profile among five HIV-infected elite controllers and five rapid progressors after recent HIV infection and one year later and from ten individuals subjected to antiretroviral therapy, five of whom were immunological non-responders (INR), before and after one year of antiretroviral treatment compared to 175 samples from HIV-negative patients. A targeted quantitative tandem mass spectrometry metabolomics approach was used in order to determine plasma metabolomics biosignature that may relate to HIV infection, pace of HIV disease progression, and immunological response to treatment.

Twenty-five unique metabolites were identified, including five metabolites that could distinguish rapid progressors and INRs at baseline. Severe deregulation in acylcarnitine and sphingomyelin metabolism compatible with mitochondrial deficiencies was observed. 6-oxidation and sphingosine-1-phosphate-phosphatase-1 activity were down-regulated, whereas acyl-alkyl-containing phosphatidylcholines and alkylglyceronephosphate synthase levels were elevated in INRs. Evidence that elite controllers harbor an inborn error of metabolism (late-onset multiple acyl-coenzyme A dehydrogenase deficiency (MADD)) was detected.

Blood-based markers from metabolomics show a very high accuracy of discriminating HIV infection between varieties of controls and have the ability to predict rapid disease progression or poor antiretroviral immunological response. These metabolites can be used as biomarkers of HIV natural evolution or treatment response and provide insight into the mechanisms of the disease.

The average period for HIV progression from acute infection to AIDS is eight years. However, elite controllers are able to naturally control HIV-1 replication and maintain adequate CD4+ T cell levels, while rapid progressors may evolve to AIDS in a period as short as two years. Furthermore, 30% of the HIV-infected population, referred to as immunological non-responders (INR), fail to increase CD4+ T cell counts by at least 30% despite being treated with antiretrovirals and achieving viral suppression for a year or more.

Metabolomics, the unbiased identification and quantification of small molecules in biological fluids, can serve as a path to the understanding of biochemical state of an organism and aid in the discovery of biomarkers. Furthermore, quantitative measurement by mass spectroscopy of specific metabolic products in plasma, urine or cells from cases compared to those from controls has begun to reveal critical differences in the products of diseased versus normal tissues for a wide variety of conditions, including prostate cancer, colon and stomach cancer, and Parkinson's disease, and HIV. In this regard, profound misbalanced functions related to energy, protein, lipid and glucose metabolism have been reported in HIV-infected individuals since recognition of the disease and introduction of ART. Increases in metabolism are reported to be present already during asymptomatic periods and can reach even higher levels during opportunistic infections. Very recently, the metabolic pathway related to the transport of the amino acid alanine was proved to be important for T cell activation; Indeed, impairments of alanine transport in CD4 T cells might contribute to HIV-1 pathogenesis through modulation of virus production, weakening of the adaptive immune response as well as enhancement of CD4 T-cell loss. Prior to the study, the inventors hypothesized that distinct individual phenotype among HIV-infected individuals will display distinct metabolomics profile.

The purpose of this study was to identify metabolites that are unique to HIV-infected individuals and to identify biomarkers that relates to HIV natural evolution and biomarkers that relate to immunological response to antiretroviral treatment using a targeted quantitative tandem mass spectrometry (MS/MS) metabolomics approach in order to gain insights into the mechanisms of HIV.

The inventors analyzed four panels of previously unthawed frozen plasma samples from HIV-infected individuals prospectively every three months using a targeted quantitative tandem mass spectrometry (MS/MS) metabolomics approach. Twenty patients were selected from a HIV recent infection cohort in Sao Paulo, Brazil. Individuals were identified as recent HIV infections using the Serologic Testing Algorithm for Recent HIV Seroconversion.

All patients were randomly selected according to their phenotype (elite controllers or rapid progressors) or their response to antiretroviral treatment. Elite controllers were defined as having a viral load below 400 copies/mL plasma after recent infection for a period of at least two years, and T+CD4 cell counts with a positive slope using linear regression.

Rapid progressors were defined as having higher viral load positive slopes and a faster decrease in CD4+T cell counts using linear regression. Selected patients were not using any concurrent medications or supplements, did not have any detected comorbidities, and did not have any laboratory abnormalities related to blood cell counts, glycose, liver, kidney or pancreatic measurements. Group A comprised samples from five elite controllers collected during recent HIV infection and after one year of follow-up. Group B used the same strategy for 5 HIV-1 rapid progressors, with samples collected during recent HIV infection and after one year of follow-up. Group C consisted of five patients who underwent antiretroviral therapy after reaching CD4+ T cell counts below 350 cells/μL, and in whom viral loads reached levels below detection limits of 50 copies/mL and CD4+ T cell counts increased to at least 30% from baseline upon treatment. Group D used the same strategy for five INR. Antiretroviral treatment on groups C and D was homogeneously comprised of an association of fixed dose combination of zidovudine and 3TC administered BID, and a QD dose of Efavirenz, according to the local Brazilian guidelines at that time. The inventors analyzed sampels from patients who experienced different paces of disease progression (Group A versus Group B) compared to patients who were either viremic (Groups B, C1 and D1), naturally aviremic (Group A), aviremic upon antiretroviral treatment (Group C2 and D2), or presented a distinct immunological response upon treatment (groups C versus D).

Metabolomic data was then analyzed. Briefly, a targeted profiling scheme was used to quantitatively screen for known small molecule metabolites using multiple reaction monitoring, neutral loss and precursor ion scans. Quantification of the metabolites of the biological sample was achieved by referencing to appropriate internal standards. The method is in conformance with 21 CFR (Code of Federal Regulations) Part 11, which implies proof of reproducibility within a given error range. The concentrations of all analyzed metabolites were reported in pM and the results were compared to tumor response rates and tumor intrinsic subtypes. This method has been used in different academic and industrial applications.

The metabolite panel is composed of 186 different metabolites: 40 acylcarnitines, 19 proteinogenic amino acids, ornithine and citrulline, 19 biogenic amines, the sum of hexoses, 76 phosphatidylcholines, 14 lyso-phosphatidylcholines and 15 sphingomyelins. Glycerophospholipids are further differentiated with respect to the presence of ester (a) and ether (e) bonds in the glycerol moiety, where two letters (aa=diacyl, ae=acyl-alkyl, ee=dia-lkyl) denote that two glycerol positions are bound to a fatty acid residue, while a single letter (a=acyl or e=alkyl) indicates the presence of a single fatty acid residue.

Lipid side chain composition is abbreviated as Cx:y, where x denotes the number of carbons in the side chain and y the number of double bonds. For example, “PC ae C38:1” denotes a plasmalogen/plasminogen phosphatidylcholine with 38 carbons in the two fatty acid side chains and a single double bond in one of them.

In addition to individual quantification, groups of metabolites related to specific functions were analyzed. Groups of AAs were computed by summing the levels of AA belonging to certain families or chemical structures depending on their functions such as essential AA, non-essential AA, glucogenic AA, total AA, branched-chain AA, Aromatic AA, glutaminolysis AA (Ala+Asp+Glu). Groups of ACs, important to evaluate mitochondrial function, were also computed by summing (Total AC, C2+C3, C16+C18, C16+C18:1, C16-OH+C18:1-OH). Groups of lipids, important to evaluate lipid metabolism, were also analyzed by summing (total LPCs, total PC aa, total PC ae, total SMs, total lipids).

Proportions among metabolites such as the Fischer's ratio, a clinical indicator of liver metabolism and function or the clinical indicators of isovaleric acidemia, tyrosinemia and urea cycle deficiency were calculated, as the ratios of branched chain amino acid (leucine+isoleucine+valine) to aromatic amino acid (tyrosine+phenylalanine), valerylcarnitine to butyrylcarnitine (C5/C4), tyrosine to serine (Tyr/Ser) respectively. A complete list of ratios reflecting enzyme activities of specific metabolic pathways have been previously described.

To unambiguously identify and quantify metabolites, stable isotope dilution-multiple reaction monitoring mass spectrometry was performed using targeted quantitative metabolomics platforms at Biocrates (Life Sciences AG, Innsbruck, Austria) in 215 plasma samples; 40 from HIV patients and 175 from controls (58 healthy volunteers, 53 colon cancer patients and 64 breast cancer patients, because the metabolic profile of activated inflammatory cells is similar to tumor cells). Multivariate profile-wide predictive models were constructed using Cross Validated Partial Least Squares Discriminant Analysis (PLS-DA). For each metabolite, the data were mean centered and scaled to unit variance. Associations between the 28 blood metabolites and HIV-1 infection were assessed using Pearson's r analysis.

The number of latent variables in each model was selected using stratified 10-fold cross validation and calculating associated R2 and Q2 statistics. The predictors were subjected to permutation testing. The results (p<5e-04) confirmed our PLS-DA analysis and revealed a clear discrimination between plasma samples from 40 samples from 20 HIV-infected individuals and 175 HIV negative counterparts employing PLS-DA and permutation testing analysis (p<5e-04 after 2000 permutations). Receiver operating characteristic (ROC) curves were determined during training and validation sets such that an accurate assessment of discriminatory ability could be made confirming the existence of highly discriminative metabolites.

Training cases were used for marker discovery and to identify any clinical variable that might be associated with a response by logistic regression analysis. Quantification of metabolite concentrations and quality control assessment was performed with the MetIQ software package (BIOCRATES Life Sciences AG, Innsbruck, Austria). Internal standards served as the reference for the metabolite concentration calculations. An Excel file was then exported, which contained sample names, metabolite names and metabolite concentration with the unit of pmol/L of plasma.

For metabolomic data analysis, log-transformation was applied to all quantified metabolites to normalize the concentration distributions. The data were uploaded into the web-based analytical pipeline MetaboAnalyst 2.0 and normalized using MetaboAnalyst normalization protocols for uni- and multivariate analysis, high dimensional feature selection, clustering and supervised classification, functional enrichment and metabolic pathway analysis. Significantly altered metabolites were defined by a T Test analysis with p-value <0.05 and FDR::;0.05.

The data were also imported to ROCCET (ROC Curve Explorer & Tester; available at ROCCET) for the generation of uni- and multivariate Receiver Operating Characteristic (ROC) curves obtained through Support Vector Machine (SVM), Partial Least Squares-Discriminant Analysis (PLS-DA) and Random Forests.

Curves were generated by Monte-Carlo cross validation (MCCV) using balanced subsampling where two thirds (2/3) of the samples were used to evaluate the feature importance. Significant features were then used to build classification models that were validated on the remaining 1/3 of the samples. The same procedure was repeated multiple times to calculate the performance and confidence interval of each model. A descriptive analysis of 28 blood metabolites and their correlation with HIV-1 infection is shown in Table 1 (above). Unsupervised multivariate analysis using Heat Map and Randon Forest classification were also conducted between cases and controls. Results demonstrated the existence of metabolites whose blood concentrations can clearly differentiate controls from patients either on acute or chronic phases. The out of the box (OOB) error, after 5000 trees, is 0.0 according Random Forest classification.

Very low concentrations of sphingomyelins and dopamine in parallel with high levels of dicarboxylicacylcarnitines, L-aspartate and many plasmalogen/plasminogen phosphatidylcholines, such as PC ae C38:1 and PC ae C40:3, were detected in the blood of HIV-1-infected individuals compared with controls.

The severe deregulation in acylcarnitine and sphingomyelin metabolism suggests that HIV infection leads to deficiencies in mitochondrial function. Therefore, ratios of certain metabolite concentrations as proxies for enzymatic activity were assembled. The proportion of esterified to free carnitines, β- and O-oxidation, and the rate-limiting step in the uptake of fatty acids into the mitochondria related to carnitine palm itoyl transferase I (CPT1) activity was then examined. The inventors also examined the SYNE2 locus because of its relation to SGPP1 (sphingosine-1-phosphate phosphatase 1) activity, a key player in the sphingosine rheostat that governs the interchange between pro-apoptotic ceramides and S1P, a well-established ligand in survival signaling.

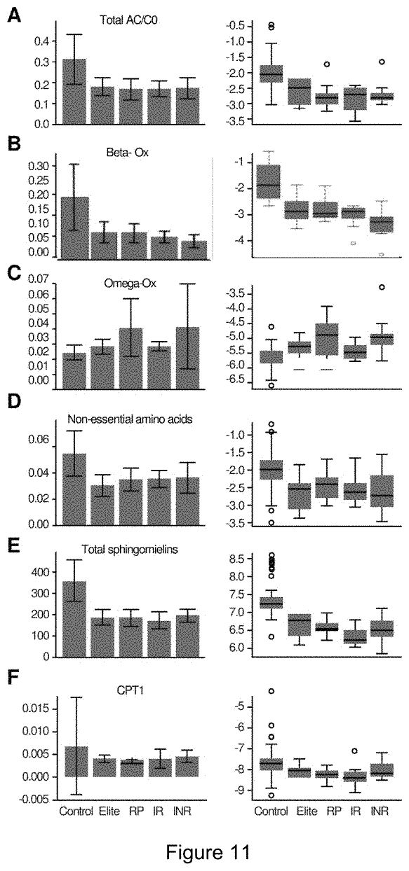

ANOVA statistical analysis confirmed our hypothesis by demonstrating that HIV infection is associated with a substantial deterioration in mitochondrial function. This conclusion is supported by a decrease in the proportion between esterified and free carnitines ((Total esterified carnitines(AC)/free carnitines (CO)) (p=9.8245E-11 and False Discovery Rate (FDR)=4.1977-10) (see FIG. 11 at A), decreased β-oxidation (p=1.3529E-13 and FDR=8.4782E-13) (see FIG. 11 at B) in parallel with increased O-oxidation (p=6.9445E-11 and FDR=3.1085E-10) (see FIG. 11 at C), and decreased uptake of fatty acids by the mitochondria (CPT1) (p=0.0016126 and FDR=0.0026136) (see FIG. 11 at F). As a consequence, the direct products of normal mitochondria, such as non-essential amino acids (p=1.5306E-47 and FDR=7.1938E-46) (see FIG. 11 at D) and sphingomyelins (p=1.1088E-18 and FDR=6.74E-19) (see FIG. 11 at E) were down-regulated in patients with HIV (see FIG. 11 at A-F). Disturbances in fatty acid oxidation (FAO), as revealed by declines in CPT1 and (3-oxidation functions, were recently reported to be very important in T cell survival and the promotion of CD8+ TM cell development. Furthermore, it has been shown that perturbations on sphingolipids and glycerophospholipids altering membrane lipid composition may impair innate immune responses. As depicted in FIG. 11 at B, β-oxidation is particularly down-regulated (p=2.5195E-8; FDR=1.1412E-7) among INR.

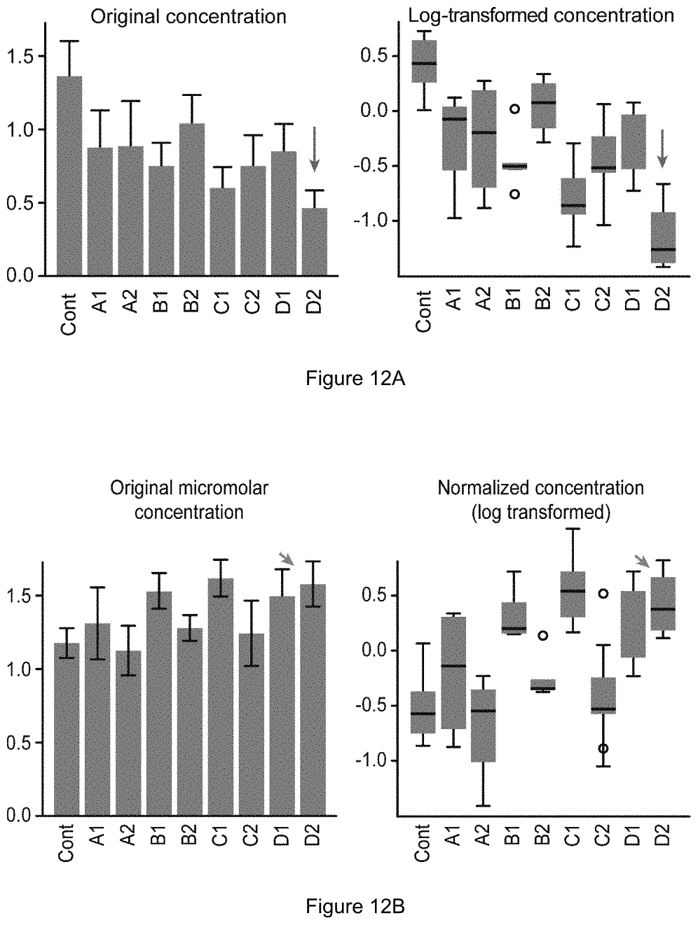

Furthermore, there was a significant decline in sphingosine-1-phosphate phosphatase 1 activity (SGPP1, SYNE2 locus) after treatment, particularly among INR, when evaluated by the ratio PC aa C28:1/PC ae C40:2 (p=8.4667E-7, -log10(p)=6.0723, FDR=1.2712E-5) (see FIG. 12A). Importantly, Sphingosine-1-Phosphate (S1 P) is involved in lymphocyte egress from lymphoid organs and bone marrow into circulatory fluids via a gradient of S1P. Because SGPP1 (SYNE2 Locus) is correlated to CD4+ T cell counts (p=0.0071195; FDR=0.16446, FIG. 12A), it is tempting to speculate the existence of a link between Sphingosine-1-Phosphate Phosphatase 1 activity and INR.

The amount of ether lipids as measured by the total acyl-alkyl-containing phosphatidylcholines to total phosphatidylcholines (AGPS) ratio was down-regulated after 1 year of follow-up in all groups but INR (p=1.1405E-5, -log10(p)=4.9429, FDR=9.6586E-5, FIG. 12B). Because ether lipids activate thymic and peripheral semi-invariant natural killer T cells known to be evolutionarily conserved lipid reactive T cells, it was hypothesized that the metabolic enzyme alkylglycerone phosphate synthase (AGPS), a critical step in the synthesis of ether lipids, could be aberrantly activated among INR, leading to impaired CD4+ T cell recovery. Thus, ether lipid biosynthesis activity after treatment vis a vis viral load level and CD4/CD8 in all patients who naturally control viremia (Elite controllers) or Immunological Responders were evaluated. The results revealed a significant negative correlation (p=8.5025E-7; FDR=1.1053E-4) between Ether Lipids (AGPS) and increasing levels of CD4 (from 160 to 1215 mm3) (PostHoc=160 >1215; 361>1215), with opposite results observed for increases in viral load (p=8.5025E-7−Log10(p)=4.9429, FDR=1.1053E-4).

In addition, the amount of ether lipids remains elevated among INR even during periods of undetectable viral load (p=1.1537E-4, FDR=3.5435E-4) when significant declines in SGPP1 (p=1.0626E-20, FDR=3.046E-19) and in p-Oxidation (p=3.3247E-5,FDR=1.0212E-4) were also observed. Lipid alterations in HIV-infected individuals receiving protease inhibitors based antiretroviral treatment determined using untargeted metabolomic profiling of plasma, has been previously linked to markers of inflammation, microbial translocation, and hepatic function, suggesting that dysregulated innate immune activation and hepatic dysfunction are occurring among HIV antiretrovirally-treated individuals. Furthermore, metabolomic profile in HIV-infected children shows hypoleptinemia and hypoadiponectinemia and is the activation of critical adipose tissue storage and function in the adaptation to malnutrition. Also, alterations in the Cerebrospinal fluid metabolome among HIV antiretrovirally-treated individuals harboring HIV-associated neuro-cognitive disorders reveal that persistent inflammation, glial responses, glutamate neurotoxicity, and altered brain waste disposal are associated with cognitive alteration.

The inventors investigated the presence of a metabolomic signature that can be used to identify “Rapid Progression” and “INR” at baseline. A combination of five different metabolites and ratios were able to accurately identify Rapid Progressors or INR at baseline with 88.89% sensitivity, 92.31% specificity, 88.89% positive predictive value and 92.31% negative predictive value (AUC=0.871; 95% CI: 0.619-1; p=0.01). During the discovery phase, the results repeatedly pointed to metabolites and ratios linked to metabolism affecting acylcarnitine hydroxylation and carboxylation as well as the catabolism of branched chain amino acids, lysine, organic acids, and tryptophan (see Table 1 above). Notably, when elevated, as seen among Elite controllers, these biochemical markers are highly suggestive of an inborn error of metabolism named late-onset multiple acyl-coenzyme A dehydrogenase deficiency (MADD, MIM#231680).

Therefore, the inventors quantified the amount of organic acids, branched chain amino acids and lysine as a diagnostic approach for MADD, in addition to using the ratio C7-DC/C8 as a proxy to analyze the activity of a MADD related enzyme, electron-transferring flavoprotein dehydrogenase (ETFDH). The results demonstrated increased levels of alpha aminoadipic acid (p=0.029658, -log10(p)=1.5279, FDR=0.078855), lysine (p=0.02768, -log10(p)=1.5578, FDR=0.075369) and Branch Chain Amino Acids (BCAA) (p=3.2721E-12, -log10(p)=11.485, FDR=1.6189E-11) among Elite controllers. Moreover, the ETFDH activity is significantly less active among Elite controllers compared to the other HIV-infected groups (T-Test=6.505E-4) and to HIV-uninfected controls (T-Test=0.0092744). Therefore, possibly an inborn error of metabolism (MADD) and its reduction of ETFDH activity, which can be asymptomatic in many individuals, relates to a control of HIV replication and a functional cure of HIV infection.

The results presented here make it clear that in addition to their utility as reliable biomarkers, metabolomic profiles of HIV-infected individuals can provide insights into mechanisms of HIV-related tissue and organ damage, and further the development of interventional strategies, such as fixing the decrease levels of dopamine seen among HIV-infected individuals in this study. Of note, low dopamine levels have been implicated in the mechanisms of psychiatric diseases such as depression and schizophrenia. As an example and corroborating the predicative abilities of the metabolic signatures identified in blood collected at baseline, of patients that years later developed specific HIV phenotypes, a recent study have been able to identify functional annotations that accurately predicted the inflammatory response of cells derived from patients suffering from inborn errors of metabolism solely on their altered membrane lipid composition.

More details concerning the foregoing study can be found in U.S. patent application Ser. No. 15/387,932, the contents of which are specifically incorporated herein, in their entirety, by reference. Because the present invention claims priority to the foregoing application (as a continuation-in-part), and therefore (by law) incorporates the contents thereof, the same will not be reproduced herein for the sake of brevity. It should be appreciated that the incorporation by reference is not limited to any particular page, column, or line from the application, and includes all signatures useful in predicting, diagnosing, and/or prognosing HIV and one's immunological response thereto. As discussed in greater detail below, these signatures, including what can be discerned therefrom, have since been found useful in assessing other viral infections and diseases, including, but not limited to COVID-19, and one's immunological response thereto.

Other Diseases (e.g., COVID-19)—Patients and Methodology

In light of the foregoing, studies were performed to identified signatures that could be used to assess other diseases, such as, for example, COVID-19. In doing so, a biological sample was obtained from a mammal, preferably a human. The biological sample preferably is blood, however, any other biological sample known to the skilled person, which allows the measurements according to the present invention is also suitable. The blood sample typically is full blood, serum or plasma, wherein blood plasma is preferred. Dried samples collected in paper filter are also accepted. Thus, the methods according to the invention typically are in vitro methods.

For the measurement of the metabolite concentrations in the biological sample a quantitative analytical method such as chromatography, spectroscopy, or mass spectrometry is employed. Targeted metabolomics were used to quantify the metabolites in the biological sample including the analyte classes of amino acids, biogenic amines, acylcarnitines, hexoses, sphingolipids and glycerophospholipids. The quantification is done using in the presence of isotopically labeled internal standards and determined by the methods as described above. A list of analytes including their abbreviations (BC codes) being suitable as metabolites to be named according to the invention is indicated in FIGS. 2-6.

In order to reach the highest capability to detect a disease using metabolomics, the present invention identified its discriminant biochemical features and ratios not only by comparing sick patients (i.e., ones having a particular disease, such as COVID-19) to healthy controls but also to a larger group of participants with other conditions.

A group of plasma samples of patients were obtained, some having certain diseases at various stages, others were from control groups. Targeted (ESI-MS/MS) Quantitative Metabolomics/Lipidomics profiling, was performed in an independent validation set with plasma samples from patients with various diseases as well as a number of controls.

Briefly, a targeted profiling scheme was used to quantitatively screen for fully annotated metabolites using multiple reaction monitoring, neutral loss and precursor ion scans. Quantification of metabolite concentrations and quality control assessment was performed with the MetIQ software package (BIOCRATES Life Sciences AG, Innsbruck, Austria) in conformance with 21 CFR (Code of Federal Regulations) Part 11, which implies proof of reproducibility within a given error range. An MS Excel file (.xls) was then generated, which contained sample identification and 186 metabolite names and concentrations with the unit of pmol/L of plasma.

For metabolomic data analysis, log-transformation was applied to all quantified metabolites to normalize the concentration distributions and uploaded into the webbased analytical pipelines MetaboAnalyst 3.0 and Receiver Operating Characteristic Curve Explorer & Tester (ROCCET) for the generation of uni- and multivariate Receiver Operating Characteristic (ROC) curves obtained through Support Vector Machine (SVM), Partial Least Squares-Discriminant Analysis (PLS-DA) and Random Forests as well as Logistic Regression Models to calculate Odds Ratios of specific metabolites. ROC curves were generated by Monte-Carlo Cross Validation (MCCV) using balanced sub-sampling where two thirds (2/3) of the samples were used to evaluate the feature importance. Significant features were then used to build classification models, which were validated on the 1/3 of the samples that were left out on the first analysis. The same procedure was repeated 10-100 times to calculate the performance and confidence interval of each model. To further validate the statistical significance of each model, ROC calculations included bootstrap 95% confidence intervals for the desired model specificity as well as accuracy after 1000 permutations and false discovery rates (FDR) calculation.

In total, 186 different metabolites were been detected being 40 acylcanitines, 19 proteinogenic aminoacids, ornithine and citrulline, 19 biogenic amines, sum of Hexoses, 76 phosphatidylcholines, 14 lyso-phosphatidylcholines and 15 sphingomyelins. See FIGS. 2-6. Glycerophospholipids are further differentiated with respect to the presence of ester (a) and ether (e) bonds in the glycerol moiety, where two letters (aa=diacyl, ae=acyl-alkyl, ee=dialkyl) denote that two glycerol positions are bound to a fatty acid residue, while a single letter (a=acyl or e=alkyl) indicates the presence of a single fatty acid residue.

Lipid side chain composition is abbreviated as Cx:y, where x denotes the number of carbons in the side chain and y the number of double bonds, e.g., “PC ae C38:1” denotes a plasmalogen/plasmenogen phosphatidylcholine with 38 carbons in the two fatty acid side chains and a single double bond in one of them.

Training cases were used for marker discovery and to identify any clinical variable that might be associated with a particular disease by logistic regression analysis. Quantification of metabolite concentrations and quality control assessment was performed with the MetIDQ® software package (BIOCRATES Life Sciences AG, Innsbruck, Austria). Internal standards serve as the reference for the metabolite concentration calculations. An xls file was then exported, which contained sample names, metabolite names and metabolite concentration with the unit of pmol/L of in plasma.

Data was then uploaded into the web-based analytical pipeline MetaboAnalyst 2.0 (www.metaboanalyst.ca) and normalized using MetaboAnalyst's normalization protocols (Xia et al 2012) for uni and multivariate analysis (see above discussion concerning normalization), high dimensional feature selection, clustering and supervised classification, functional enrichment as well as metabolic pathway analysis.

Data was also imported to ROCCET (ROC Curve Explorer & Tester) available at http://www.roccet.ca/ROCCET/for the generation of uni and multivariate Receiver Operating Characteristic (ROC) curves obtained through Support Vector Machine (SVM), Partial Least Squares-Discriminant Analysis (PLS-DA) and Random Forests.

Curves were generated by Monte-Carlo cross validation (MCCV) using balanced subsampling where two thirds (2/3) of the samples were used to evaluate the feature importance. Significant features were then used to build classification models, which were validated on the 1/3 of the samples that were left out. The same procedure was repeated multiple times to calculate the performance and confidence interval of each model.

Other Diseases (e.g., COVID-19)—Signatures

A descriptive analysis of 28 blood metabolites and their correlation with COVID-19 infection is shown in Table 1 (see above). Very low concentrations of sphingomyelins and dopamine in parallel with high levels of dicarboxylicacylcarnitines, L-aspartate and many plasmalogen/plasminogen phosphatidylcholines, such as PC ae

C38:1 and PC ae C40:3, were detected in the blood of COVID-19-infected individuals compared with controls.

The severe deregulation in acylcarnitine and sphingomyelin metabolism suggests that viral infection (e.g., COVID-19) leads to deficiencies in mitochondrial function. Therefore, the inventors assembled ratios of certain metabolite concentrations as proxies for enzymatic activity. They examined the proportion of esterified to free carnitines, β- and O-oxidation, and the rate-limiting step in the uptake of fatty acids into the mitochondria related to carnitine palmitoyl transferase I (CPT1) activity. They also examined the SYNE2 locus because of its relation to SGPP1 (sphingosine-1-phosphate phosphatase 1) activity, a key player in the sphingosine rheostat that governs the interchange between pro-apoptotic ceram ides and S1 P, a well-established ligand in survival signaling.

ANOVA statistical analysis confirmed our hypothesis by demonstrating that the viral infection is associated with a substantial deterioration in mitochondrial function. This conclusion is supported by a decrease in the proportion between esterified and free carnitines ((Total esterified carnitines(AC)/free carnitines (CO)) (p=9.8245E-11 and False Discovery Rate (FDR)=4.1977-10) (see FIG. 11 at A), decreased β-oxidation (p=1.3529E-13 and FDR=8.4782E-13) (see FIG. 11 at B) in parallel with increased O-oxidation (p=6.9445E-11 and FDR=3.1085E-10) (see FIG. 11 at C), and decreased uptake of fatty acids by the mitochondria (CPT1) (p=0.0016126 and FDR=0.0026136) (see FIG. 11 at F). As a consequence, the direct products of normal mitochondria, such as non-essential amino acids (p=1.5306E-47 and FDR=7.1938E-46) (see FIG. 11 at D) and sphingomyelins (p=1.1088E-18 and FDR=6.74E-19) (see FIG. 11 at E) were down-regulated in patients with viral infections (see FIG. 11 at A-F). Disturbances in fatty acid oxidation (FAO), as revealed by declines in CPT1 and β-oxidation functions, were recently reported to be very important in T cell survival and the promotion of CD8+ TM cell development. Furthermore, it has been shown that perturbations on sphingolipids and glycerophospholipids altering membrane lipid composition may impair innate immune responses. As depicted in FIG. 11 at B, β-oxidation is particularly down-regulated (p=2.5195E-8; FDR=1.1412E-7) among INR.

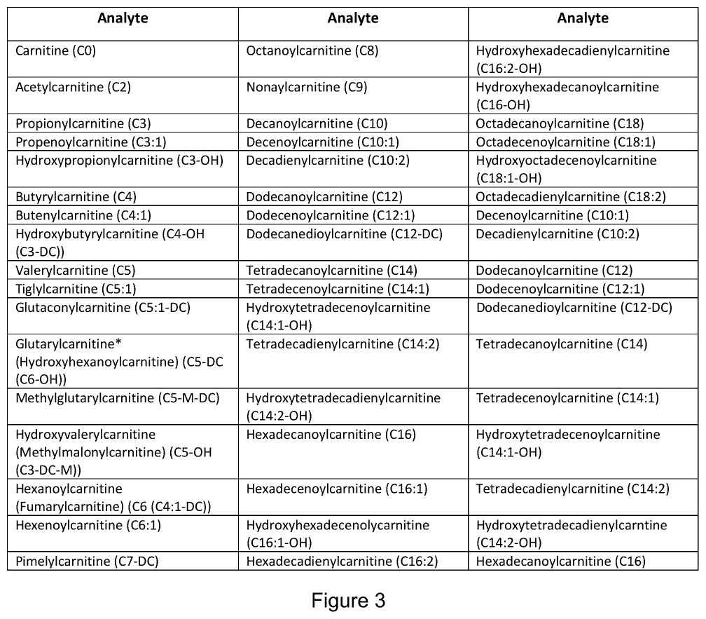

Furthermore, there was a significant decline in sphingosine-1-phosphate phosphatase 1 activity (SGPP1, SYNE2 locus) after treatment, particularly among INR, when evaluated by the ratio PC aa C28:1/PC ae C40:2 (p=8.4667E-7, -log10(p)=6.0723, FDR=1.2712E-5) (see FIG. 12A). Importantly, Sphingosine-1-Phosphate (S1 P) is involved in lymphocyte egress from lymphoid organs and bone marrow into circulatory fluids via a gradient of S1P. Because SGPP1 (SYNE2 Locus) is correlated to CD4+ T cell counts (p=0.0071195; FDR=0.16446, FIG. 3), it is tempting to speculate the existence of a link between Sphingosine-1-Phosphate Phosphatase 1 activity and INR.

The amount of ether lipids as measured by the total acyl-alkyl-containing phosphatidylcholines to total phosphatidylcholines (AGPS) ratio was down-regulated after 1 year of follow-up in all groups but INR (p=1.1405E-5, -log10(p)=4.9429, FDR=9.6586E-5, FIG. 12B). Because ether lipids activate thymic and peripheral semi-invariant natural killer T cells known to be evolutionarily conserved lipid reactive T cells, we hypothesized that the metabolic enzyme alkylglycerone phosphate synthase (AGPS), a critical step in the synthesis of ether lipids, could be aberrantly activated among INR, leading to impaired CD4+T cell recovery.

The inventors therefore evaluated ether lipid biosynthesis activity after treatment vis a vis viral load level and CD4/CD8 in all patients who naturally control viremia (Elite controllers) or Immunological Responders. The results revealed a significant negative correlation (p=8.5025E-7; FDR=1.1053E-4) between Ether Lipids (AGPS) and increasing levels of CD4 (from 160 to 1215 mm3) (PostHoc=160 >1215; 361 >1215), with opposite results observed for increases in viral load (p=8.5025E-7 Log10(p)=4.9429, FDR=1.1053E-4). In addition, the amount of ether lipids remains elevated among INR even during periods of undetectable viral load (p=1.1537E-4, FDR=3.5435E-4) when significant declines in SGPP1 (p=1.0626E-20, FDR=3.046E-19) and in β-Oxidation (p=3.3247E-5,FDR=1.0212E-4) are also observed. Lipid alterations in viral infected individuals receiving protease inhibitors based antiretroviral treatment determined using untargeted metabolomic profiling of plasma, has been previously linked to markers of inflammation, microbial translocation, and hepatic function, suggesting that dysregulated innate immune activation and hepatic dysfunction are occurring among viral antiretrovirally-treated individuals. Furthermore, metabolomic profile in viral-infected children shows hypoleptinemia and hypoadiponectinemia and is the activation of critical adipose tissue storage and function in the adaptation to malnutrition. Also, alterations in the Cerebrospinal fluid metabolome among viral antiretrovirally-treated individuals harboring viral-associated neuro-cognitive disorders reveal that persistent inflammation, glial responses, glutamate neurotoxicity, and altered brain waste disposal are associated with cognitive alteration.

The inventors investigated the presence of a metabolomic signature that can be used to identify “Rapid Progression” and “INR” at baseline. A combination of five different metabolites and ratios were able to accurately identify Rapid Progressors or INR at baseline with 88.89% sensitivity, 92.31% specificity, 88.89% positive predictive value and 92.31% negative predictive value (AUC=0.871; 95% CI: 0.619-1; p=0.01). During the discovery phase, the results repeatedly pointed to metabolites and ratios linked to metabolism affecting acylcarnitine hydroxylation and carboxylation as well as the catabolism of branched chain amino acids, lysine, organic acids, and tryptophan (see Table 1 above). Notably, when elevated, as seen among Elite controllers, these biochemical markers are highly suggestive of an inborn error of metabolism named late-onset multiple acyl-coenzyme A dehydrogenase deficiency (MADD, MIM#231680)

Therefore, the amount of organic acids, branched chain amino acids and lysine as a diagnostic approach for MADD[32], in addition to using the ratio C7-DC/C8 as a proxy to analyze the activity of a MADD related enzyme, electron-transferring flavoprotein dehydrogenase (ETFDH) were quantified. The results demonstrated increased levels of alpha aminoadipic acid (p=0.029658, -log10(p)=1.5279, FDR=0.078855), lysine (p=0.02768, -log10(p)=1.5578, FDR=0.075369) and Branch Chain Amino Acids (BCAA) (p=3.2721E-12,-log10(p)=11.485, FDR=1.6189E-11) among Elite controllers. Moreover, the ETFDH activity is significantly less active among Elite controllers compared to the other viral-infected groups (T-Test=6.505E-4) and to viral-uninfected controls (T-Test=0.0092744). Therefore, possibly an inborn error of metabolism (MADD) and its reduction of ETFDH activity, which can be asymptomatic in many individuals, relates to a control of viral replication and a functional cure of viral infection.

The results presented here make it clear that in addition to their utility as reliable biomarkers, metabolomic profiles of viral-infected individuals can provide insights into mechanisms of viral-related tissue and organ damage, and further the development of interventional strategies, such as fixing the decrease levels of dopamine seen among infected individuals in this study. Of note, low dopamine levels have been implicated in the mechanisms of psychiatric diseases such as depression and schizophrenia.

While the foregoing study was aimed at viral infections, in general, additional studies have been performed to identify signatures useful in assessing patient's with other viral infections, such as COVID-19. With respect to COVID-19, and other viral infections, the inhibition of viral replication has been a major focus of research and development. Another approach is the development of vaccines that alert and prepare the human immune system to respond to viral antigens. This augments immune response, allowing vaccinated individuals to eliminate viruses before they can invade cells and propagate. The principal of vaccination rests upon the premise that an individual's immune system when appropriately primed can and will marshal a virucidal response.

Patients with primary and secondary immune deficiencies can receive inactive vaccines (recombinant, subunit, toxoid, polysaccharride, conjugated polysaccharide, CPV, TDP, etc.) but these individuals do not generate a robust immune response. Live attenuated vaccines are contraindicated in immunocompromised individuals as they can result in life-threatening iatrogenic infections. Thus, each individual's immune competence (the underlying capacity of the immune system to provide protection against infecting organisms) is fundamental to their success in controlling and surviving infectious diseases.

The COVID-19 pandemic identified several factors associated with poor prognosis, including advanced age, cardiovascular disease, diabetes and obesity yet otherwise healthy individuals with no co-morbidities also succumbed and die from infection. Furthermore, many individuals with serological evidence of COVID-19 exposure had minimal or no clinical features of the disease. This suggests that response and survival following COVID-19 infection reflects each individual's immune response, yet there are no tests available today that can define an individual's likelihood of clinical disease, morbidity or mortality from this disease.

The incapacity of medical scientists to define populations at the risk of severe COVID-19-related illness and death led to the unprecedented lockdown of the US economy, while all people, young and old, healthy and ill were equally mandated to “shelter-in-place” costing the American economy over 20 million jobs and untold trillions of dollars.

In one embodiment, this invention applies metabolic measures of immune competence using metabolite ratios to define each individual's capacity to marshal an effective immune response against offending organisms. Unlike CD4 counts, CD4/CD8 ratios, viral load titers or serological measures that are used as surrogate measures of the disease state, this invention defines each individual's physiological capacity to generate an effective immune response, marshal an appropriate defense and go on to survive infection.

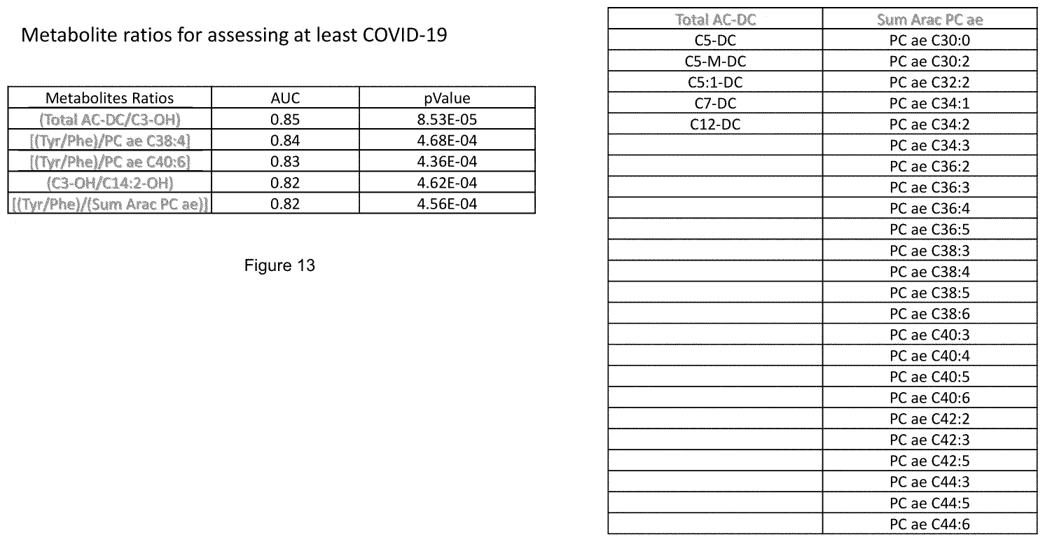

The metabolite ratios depicted in FIG. 13 have been shown to define HIV-infected individuals who do not progress from HIV infection to AIDS. These ratios have also been shown to predict for survival in individuals with hematologic neoplasms. Viral infections (CMV, HSV, VZV, RSV, etc.) are an important cause of morbidity and mortality in patients with hematological neoplasms, with their incidence and severity correlating directly with the intensity and duration of T-cell mediated immune suppression. While it is well known that HIV infection predisposes individuals to aggressive hematologic malignancies, it has now been shown that persons with hematological malignancies have a statistically higher morbidity and mortality following COVID-19 infection. This supports commonalities between viral infection, hematologic malignancy and the capacity of each individual to mount an effective immune response.

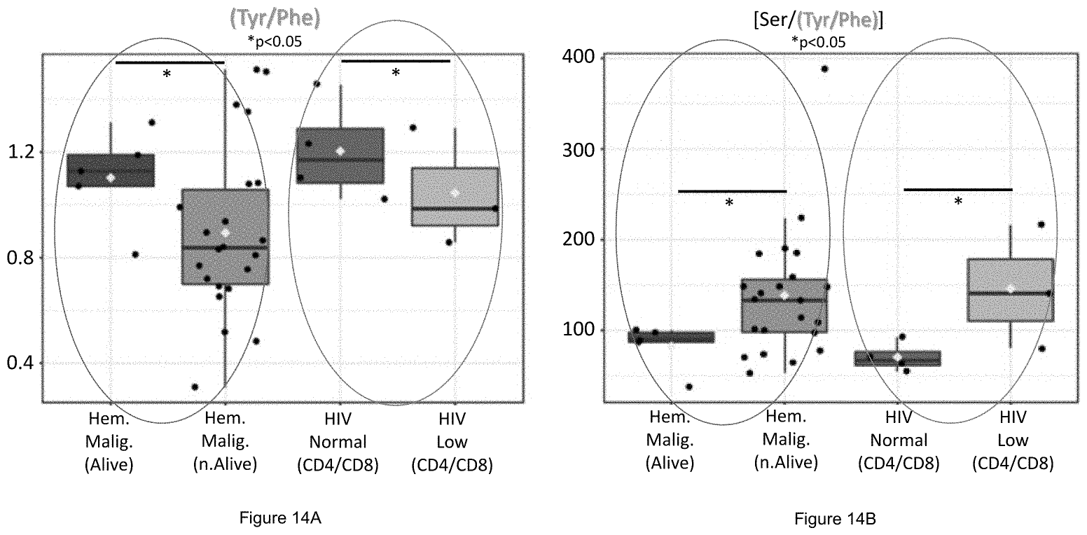

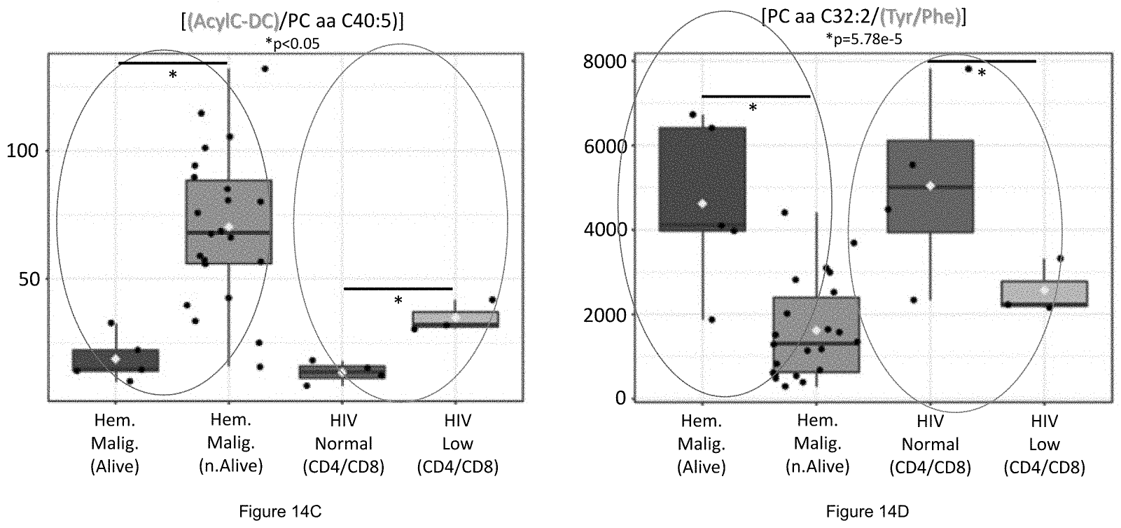

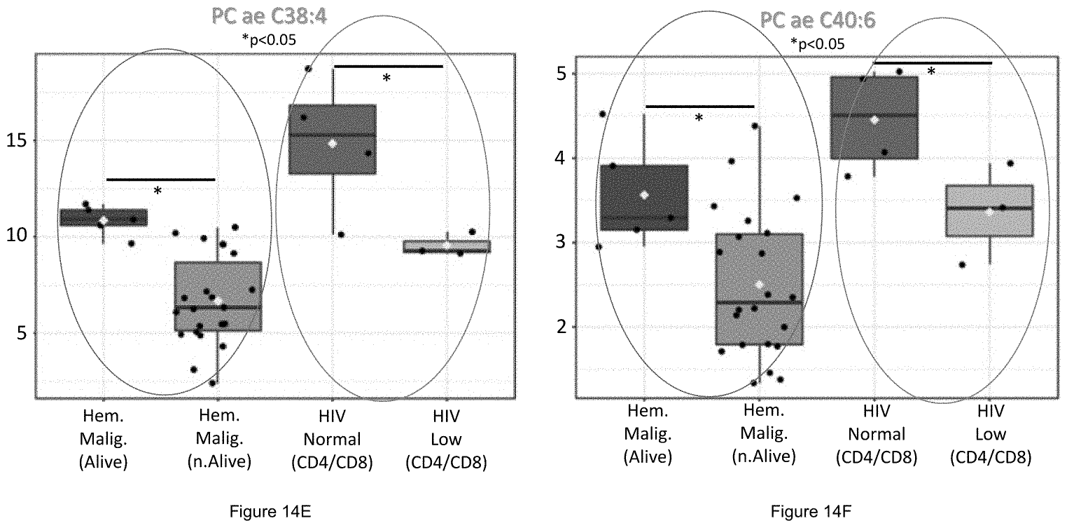

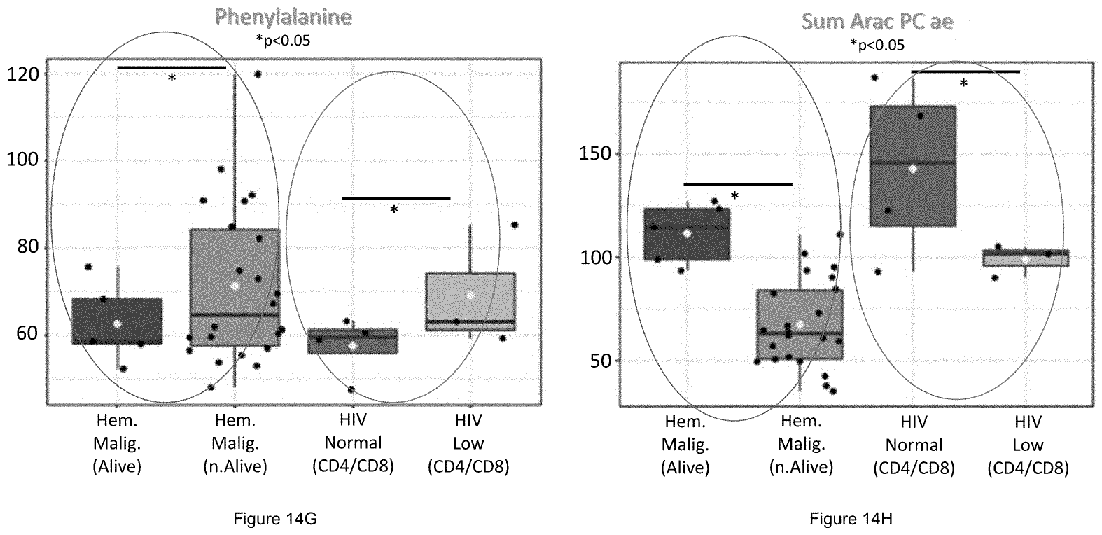

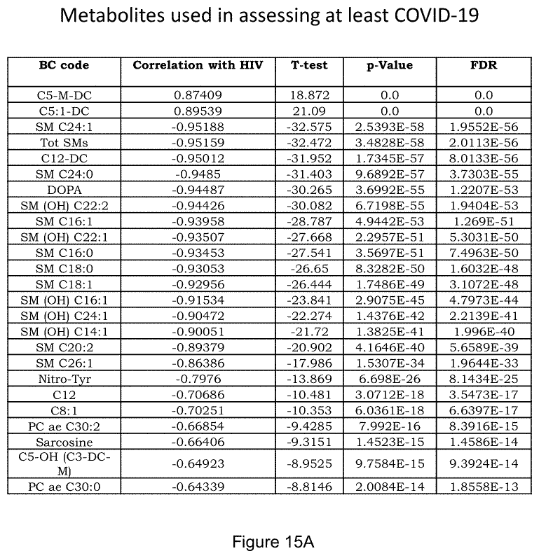

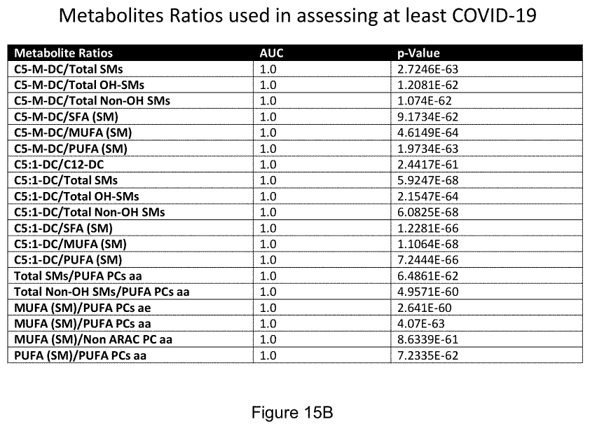

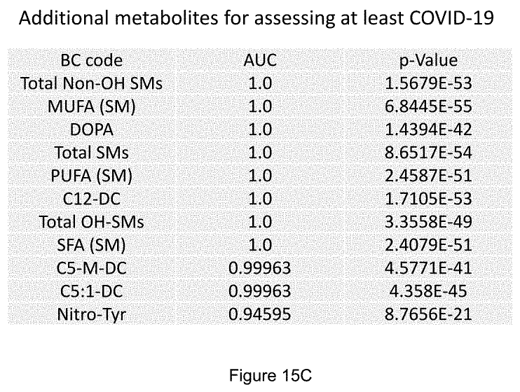

The data provided in FIGS. 14A-H, using metabolites described in FIGS. 15A-C, confirm the nexus between the survival of individuals with viral infections and survival of individuals with blood borne cancers reflecting the level of immune competence measured by the invention. In particular, FIGS. 14A-H show the metabolites and ratios that are important for the prediction between HIV cases with good (HIV normal CD4/CD8) and bad (HIV low CD4/CD8) response to antirretroviral drugs. Of importance, the same pattern of ups and downs are seen in cases of Hematological Malignancies that were dead or alive during a five year period.

By doing these comparisons, the inventors are able to show that the same metabolites and/or ratios that predict worse outcomes in HIV patients, are able to discriminate cases of cancer with bad and good prognosis. Importantly, from the perspective of the natural history of disease, HIV patients are known to be at elevated risk to develop Hematological Malignancies and our biochemical results are fully supporting these epidemiological observations. Viral infections are important causes of morbidity and mortality for patients with a hematological malignancy. The difference in incidence and outcome of viral infections among patient groups is wide, but dependent upon the intensity and duration of T-cell-mediated immune suppression. Infections caused by cytomegalovirus (CMV), herpes simplex virus (HSV), varicella-zoster virus (VZV), respiratory syncytial virus (RSV), parainfluenza viruses and influenza viruses have been intensely studied, yet newly recognized aspects of these viral infections including late CMV infection; the emergence of new viral pathogens (human herpesvirus-6, BK virus, adenovirus, and human metapneumovirus) and more recently, the COVID-19 invention, very likely share the same metabolomic pattern as HIV. Indeed, subjects with hematological cancers had more severe COVID-19 and more deaths compared with hospitalized health care providers with COVID-19.

The inventors have discovered metabolomic signatures (e.g., ratios, sums, etc.) that are directly related to CD4 and CD8 values, which are major controllers of our immunity independently of any type of virus infection, as well as cancer. As such, the inventors have discovered that virtually identical signatures can also be used to assess a patient with respect to COVID-19.

The plasma-based, mass spectrometer-measured immune signatures described in this invention define each individual's immune competence score that can be applied before, during or after infection to provide prognostic information regarding the likelihood of clinical infection following exposure as well as the severity of illness and the likelihood of illness-related mortality.

While the equations shown in FIG. 13, and the metabolite (or ratios) illustrated in FIGS. 14A-H, and identified in FIGS. 15A-C, are important aspects of the COVID-19 signature, of particular importance is the ratio of Tyr/Phe (see FIG. 14A) and the Sum Arac PC ae (see FIG. 14A).

The term “Sum Arac PC ae” is related to the summation of each individual molar concentration of the lipids described in this table. The term “Arac” stems for “Arachdonic” used to describe lipids containing 36 or more carbon units which are the majority here. The term “PC ae” is a short version of “Phosphatidylcholine (PC) containing Acyl-Ether bonds.” These lipids are also known as ether-lipids and very little is known about their properties. The inventors have discovered their connection to CD4 and CD8, one of the major pathways controlled by our inborn/innate immunity.

CD4 helper/inducer cells and CD8 cytotoxic/suppressor cells are two phenotypes of T lymphocytes, characterized by distinct surface markers and functions that mostly reside in lymph nodes but also circulate in the blood. The normal CD4/CD8 ratio in healthy hosts is poorly defined. Ratios between 1.5 and 2.5 are generally considered normal; however, a wide heterogeneity exists because sex, age, ethnicity, genetics, exposures, and infections may all impact the ratio. Normal ratios can invert through isolated apoptotic or targeted cell death of circulating CD4 cells, expansion of CD8 cells, or a combination of both phenomena. A low or inverted CD4/CD8 ratio is an immune risk phenotype and is associated with altered immune function, immune senescence, and chronic inflammation in both HIV-infected and uninfected populations.

The prevalence of an inverted CD4/CD8 ratio increases with age. An inverted ratio is seen in 8% of 20- to 59-year-olds and in 16% of 60- to 94-year-olds. Women across all age groups are less likely to have an inverted ratio than their male counterparts. Age- and hormone-related atrophy of the thymus is theorized to explain the differences between populations. Hormonal influence on the ratio is supported by a correlation between low plasma estradiol levels, high circulating CD8, and low CD4/CD8 ratios in women with premature ovarian failure.

In the HIV negative population, a low CD4/CD8 immune risk phenotype reflects immune senescence, is associated with wide-ranging pathology, and may also predict morbidity and mortality. Irreversible disruption of self-immunologic tolerance to endogenous antigens is a hallmark of autoimmune disease. In this setting of immune dysfunction, an abnormal CD4/CD8 ratio can emerge. Furthermore, while an abnormal ratio is not uniformly present in all autoimmune diseases, a decreased CD4/CD8 ratio is consistently seen in systemic lupus erythematosus. A low CD4/CD8 ratio reflects p-cell destruction and may predict diabetes diagnoses in first-degree relatives of type 1 diabetic probands. In a population study of solid neoplasms, an inverted CD4/CD8 ratio is associated with metastatic disease as compared with cancer patients without metastasis. Moreover, following acute myocardial infarction and cardiopulmonary resuscitation, a fixed low CD4/CD8 ratio is a poor prognostic sign. Despite these associations, it is important to acknowledge that the presence of a low CD4/CD8 ratio is not clearly the cause or the effect of the above pathology. This acknowledgment is further highlighted by the presence of a low ratio in conditions outside the umbrella of traditional organic pathology, including an association between low ratios and pessimists. Conflicting literature exists regarding the use of an inverted CD4/CD8 ratio (<1.0) as a predictor for mortality in elderly HIV-negative populations.

Two longitudinal cohorts of elderly Swedish individuals demonstrated that an inverted ratio (<1.0) was associated with frailty and mortality. These studies helped define the immune risk phenotype and raised the possibility of using the CD4/CD8 ratio as a biomarker to stratify risk in elderly populations. Later cohort studies in Spain and the United Kingdom found that while a low CD4/CD8 ratio was associated with time to death in unadjusted analyses, no association between the ratio and morbidity was found in multivariable analyses. Moreover, a recent cross-sectional study of frailty and prospective cohort study of morbidity in residents of Canadian nursing homes found that greater percentages of central memory CD8+T cells were more predictive of increased frailty than other immune phenotypes, including an inverted CD4/CD8 ratio. Thus, the CD4/CD8 ratio may not be a marker for morbidity and/or mortality in all populations. Hence the need for the presention inveniton, which provides a better approach to assess (e.g., prognose) individuals for COVID-19.

Determining and Providing Results

The invention may involve a patient visiting a doctor, clinician, technician, nurse, etc., where blood or a different sample is collected. The sample would then be provided to a laboratory for analysis, as discussed above (e.g., mass spectrometry, log-transformation, comparisons, etc.). In another embodiment, a kit can be used to obtain the sample, where the kit is made available to the patient via a medical facility, a drug store, the Internet, etc. In this embodiment, the kit may include one or more wells and one or more inserts impregnated with at least one internal standard. The kit can be used to gather the sample from a patient, where the sample is then provided to a laboratory for analysis.

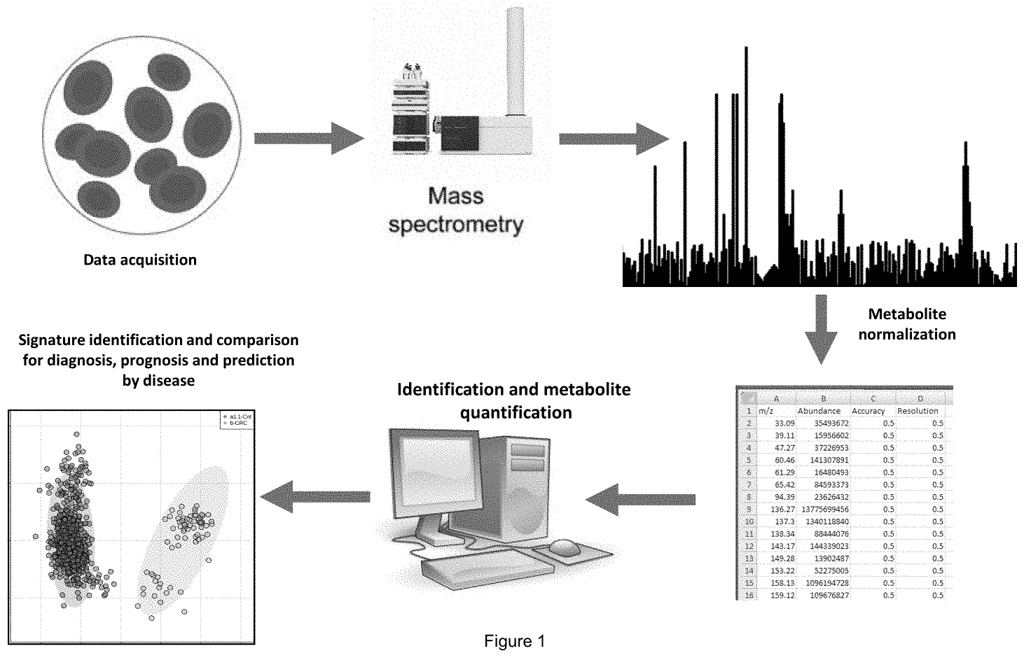

For example, as shown in FIG. 1, peripheral blood may collected into EDTA-anticoagulant tubes. Plasma is isolated by centrifugation. Plasma samples may then be submitted to a p180 AbsolutelDQ kit for extraction and processing. In one embodiment, prepared samples will then undergo liquid chromatography (LC) followed by Flow Injection Analysis (FIA) by tandem Mass Spectrometry (MS/MS) (i.e., metabolite extraction). The extracted data is then processed using computer software. For example, the data acquired may then be normalized (e.g., via log-transformation) and stored in a database that includes at least (i) patient identification, (ii) metabolite name, and (iii) quantification. If this data is on known individuals (individuals with known conditions), then it can be analyzed to determine signatures that can be used to assess a particular disease. If, however, the data is on a patient whose condition is unknown, then it can be compared to known signatures (e.g., stored in memory) to screen for, diagnose, prognose, and treat the patient.

It should be appreciated that the present invention is not limited to normalizing a quantified metabolite. In other words, other processes discussed herein and/or generally known to those skilled in the art may be performed either before or after normalization. It should also be appreciated that while certain processes can be performed manually, most (if not all) should preferably be performed using software, where initial results (data post mass spectrometry, post normalization), are stored in memory, presented on a display (e.g., computer monitor, etc.) and/or printed. The initial results can then be compared to known “signatures” for different diseases, where similarities and differences are used to screen for, diagnose, prognose, treat, etc. a particular disease. It should be appreciated that the sample may be assessed for a particular disease, or for multiple diseases, depending on the patient's sex, age, etc. Thus, the software could be used to assess a particular disease or assess at least one disease from a plurality of diseases.

It should further be appreciated that the “comparing” step can be performed by (i) software, (ii) a human, or (iii) both. For example, with respect to the prior, a computer program could be used to compare sample results to known signatures and to use differences and/or similarities thereof to assess at least one disease, and provide diagnosis, prognosis, and/or treatment for the same. Alternatively, in the second embodiment, a technician could be used to compares sample results to known signatures (or aspects thereof) and make a diagnosis, prognosis, and/or treatment decision based on perceived similarities and/or differences. Finally, with respect to the latter, a computer program could be used to plot (e.g., on a computer display) sample results alongside known signatures (e.g., signatures of healthy patients, signatures of unhealthy patients, life expectancies, etc.). A technician could then view the same and make at least one diagnosis, prognosis, treatment recommendation, etc. based on similarities and/or differences in the plotted information.

Bottom line, it is the differences and/or similarities between known signatures that allows a disease to be assessed, whether that assessment is automated (e.g., performed by a computer), performed manually (e.g., done by a human), or a combination of the two.

Results (e.g., assessments) are then provided to the patient directly (e.g., via mail, an electronic communication, etc.) or via the patient's doctor, and can include screening information, diagnosis information, prognosis information, and treatment information.

In particular, the invention can be used to distinguish a sample that is infectious (positive) from one that is normal (negative). If it is positive, then the invention can further be used to distinguish, a first virus from a second virus, etc. Once the disease is identified, the invention can then be used to define the disease, by degree, progression, etc. This can be done using terminology (e.g., lethal, etc.), at least one scale (e.g., 1-10, 1-100, A-F, etc.), where one end of the scale is low grade (e.g., asymptomatic) and the other end is high grade (lethal), or other visual forms (e.g., color coded, 2D or 3D model, etc.).

The invention can also be used to provide a prognosis. For example, in COVID-19, once the disease is identified (or even prior to the patient coming down with the disease), the invention can be used to provide gradations within the signature (or signatures), subcategorizing the patient into one that is likely to survive, likely to be asymptomatic, or likely to die. Again, prognosis could be provided using terminology (e.g., low risk, medium risk, high risk, etc.), at least one scale, or other visual forms.

For example, certain individuals will contract COVID-19 yet respond well (e.g., few if any symptoms). Others will not respond so well, be symptomatic, develop conditions (e.g., pneumonia), some of which could lead to death. Indicators that a patient is not responding well to a COVID-19 infection include the patient's arterial oxygen pressure (partial pressure of oxygen, or PaO2). At sea level, a normal arterial oxygen pressure is between 75 and 100 mmHg. Thus, a patient that responds well to an infection may not experience a drop in arterial oxygen pressure (i.e., their pressure may remain at or above 75 mmHg), whereas a patient that does not respond well, their oxygen pressure may drop (e.g., below 75 mmHg).

It should be appreciated that other indicators (e.g., objective indicia) of COVID-19 (or other diseases) are within the spirit and scope of the present invention. For example, a person that responds well may be asymptomatic (e.g., does not experience (or has minor experiences of) cough, shortness of breath, fever, loss of smell, loss of taste, etc.), where a person that responds poorly may have one or more of the foregoing symptoms and suffer from one or more condition, such as diabetes mellitus, hypoxemic respiratory failure, hypertension, acute respiratory distress syndrome (ARDS), acute-onset hypoxemia (ratio of arterial oxygen to the fraction of inspired oxygen <300), abnormal chest scans (e.g., bilateral pulmonary opacities, etc.), lymphocytopenia (e.g., lymphocyte count below 1000 per cubic millimeter), hyperlactatemia (e.g., greater than 1 mmol/L), elevated hepatic enzymes, elevated troponin concentrations, low white cell counts, elevated C-reactive proteins, etc. It should also be appreciated that a person may not merely respond well or poorly (i.e., binarily), but may respond better than others, more poorly than others, have a range of responses (e.g., great, good, fair, poor, etc., a scale from 1-5, etc.). For example, an individual may be provided with their immune competence score, which, at the very least, will identify the individual as good or worse, but may provide more delineation (e.g., likely to be asymptomatic, likely to experience some symptoms but recover without therapy, likely to experience many symptoms but recover with therapy, unlikely to survive, etc.).

Not only can the present invention be used to determine life expectancy and remission rate, it can also be used to determine treatment, or viability of treatment (another form of prognosis). This could be a likelihood to respond to therapy (e.g., hormonal, radiation, chemotherapy, etc.), which again could be provided using terminology, at least one scale, or other visual forms.

Thus, by way of example, the present invention may be used to determine (i) a high likelihood that a patient harbors a virus (diagnosis), (ii) a high likelihood that the virus is COVID-19 (diagnosis), (iii) response to therapy (e.g., antiretroviral drugs, ventilator, etc.) (prognosis), and (iv) immune competency (e.g., likelihood of being asymptomatic, likely to survive, etc.) (prognosis). Clearly this is exemplary, and other diseases, sub-categorizations, prognosis, and treatments can be identified (predicted) using the present invention.

The invention can also be used to screen for diseases. Medical screening is the systematic application of a test or inquiry to identify individuals at sufficient risk of a specific disorder to benefit from further investigation or direct preventative action (these individuals not having sought medical attention on account of symptoms of that disorder). The present invention uses metabolic signatures to screen for diseases in populations who are considered at risk. For COVID-19, this may be people over 65, with diabetes, heart conditions, or other risk factors.

It should be appreciated that while several examples have been provided as to what the present invention can discern from a blood sample (or the like), the present invention is not so limited, and other types of diagnosis and prognosis, including treatments, are within the spirit and scope of the present invention. For example, COVID-19 may be identified by severity, stage, etc. It may also be identified by its prognosis (e.g., good response, etc.). Those skilled in the art will understand that similar classifications can be provided for other diseases, where such classification are generally known to those skilled in the art. All such classifications, for both diagnosis and prognosis, are within the spirit and scope of the present invention.

As shown in FIG. 1, once a sample has been received and processed (e.g., processed using techniques like the one used to identify the signatures in the first place, such as mass spectrometry (to quantify metabolites), log-transformation (or other mathematical manipulation to normalize the data), etc.), the initial results (e.g., metabolites and/or sets thereof) can then be compared to signatures (or portions thereof) that have been identified (by the inventors) as useful in assessing at least one disease. The signatures may be stored in memory, and the initial data (i.e., processed sample) may be compared to at least one signature either manually (e.g., by viewing the sample, or initial results thereof, against known signatures), automatically (e.g., using a computer program to discern differences and/or similarities between the sample, or initial results thereof, and known signatures), or both (e.g., a program determines at least one diagnosis/prognosis and a technician reviews the data to validate the same). Based on the results (i.e., comparison results), at least one diagnosis and/or prognosis, which may or may not include treatment, is identified and provided to the patient.

### CONCLUSION

Having thus described several embodiments of a system and method for using new biomarkers for assessing different diseases, it should be apparent to those skilled in the art that certain advantages of the system and method have been achieved. It should also be appreciated that various modifications, adaptations, and alternative embodiments thereof may be made within the scope and spirit of the present invention. The invention is solely defined by the following claims.

What is claimed is:

1. A method for prediction of immunological response of a human patient with COVID-19, comprising:
using a technology selected from chromatography, spectroscopy, and spectrometry to quantify a plurality of metabolites included in a blood sample obtained from said human patient, including at least Tyrosine and Phenylalanine;
normalizing at least said Tyrosine and said Phenylalanine, as quantified using said technology;
comparing at least a result of an equation comprising at least a first ratio of said Tyrosine and said Phenylalanine, as normalized, to at least one predetermined value to determine at least one level of similarity therebetween; and
using said at least one level of similarity to at least predict said human patient's response to having COVID-19, said response being one of a good response or a worse response;
wherein said good response comprises an arterial oxygen pressure above 75 mmHg at sea level and a high likelihood that said patient will either be asymptomatic or respond well to therapy and a worse response comprises an arterial oxygen pressure below 75 mmHg at sea level and a low likelihood that said patient will either be asymptomatic or respond well to therapy.

2. The method of claim 1, wherein said step of quantifying and normalizing said Tyrosine and said Phenylalanine further comprises the step of quantifying and normalizing at least one Phosphatidylcholine with Acyl-Alkyl Residue.

3. The method of claim 2, wherein said step of quantifying and normalizing said Tyrosine and said Phenylalanine further comprises the step of quantifying and normalizing a plurality of Phosphatidylcholine with Acyl-Alkyl Residue.

4. The method of claim 1, wherein said first ratio comprises said Tyrosine to said Phenylalanine.

5. The method of claim 3, wherein said plurality of Phosphatidylcholine with Acyl-Alkyl Residue are selected from group of Arachdonic Phosphatidylcholine with Acyl-Alkyl Residue.

6. The method of claim 5, wherein said equation further comprises a summation of said plurality of Phosphatidylcholine with Acyl-Alkyl Residue.

7. The method of claim 4, wherein said step of quantifying and normalizing said Tyrosine and said Phenylalanine further comprises the step of quantifying and normalizing a plurality of Phosphatidylcholine with Acyl-Alkyl Residue.

8. The method of claim 7, wherein said plurality of Phosphatidylcholine with Acyl-Alkyl Residue are selected from a group of Arachdonic Phosphatidylcholine with Acyl-Alkyl Residue, and said equation further comprises a summation of said plurality of Phosphatidylcholine with Acyl-Alkyl Residue.

9. The method of claim 8, wherein said equation further comprises a second ratio, said first ratio comprising Tyrosine to said Phenylalanine and said second ratio comprises said first ratio to said summation of said plurality of Phosphatidylcholine with Acyl-Alkyl Residue.

10. The method of claim 1, wherein said step of normalizing at least said Tyrosine and Phenylalanine further comprises using at least a log-transformation to normalize at least said Tyrosine and Phenylalanine.

11. The method of claim 1, wherein said response further comprises an immune competence score, said score comprising a value between a lower limit value and a higher limit value.

12. The method of claim 11, wherein said score comprises one of at least three values, a first one of which corresponds to poor, a second one of which corresponds to fair, and a third one of which corresponds to good.

13. The method of claim 11, wherein said score comprises at least a likelihood of survival.

14. A system for prediction of immunological response of a human patient with COVID-19, comprising:
a computing system comprising at least one memory device for storing machine readable instructions adapted to perform the steps of:
receive a plurality of quantified metabolites from a sample provided by said human patient, including at least Tyrosine and Phenylalanine;
normalize said plurality of quantified metabolites;
compare at least a result of an equation comprising at least a first ratio of said Tyrosine and said Phenylalanine, as normalized, to at least one predetermined value to determine at least one level of similarity therebetween; and
use said at least one level of similarity to at least predict said human patient's response to having COVID-19, said response being one of a good response and a poor response;
wherein said good response comprises an arterial oxygen pressure above 75 mmHg at sea level and therefore a high likelihood that said patient will either be asymptomatic or respond well to therapy and a worse response comprises an arterial oxygen pressure below 75 mmHg at sea level and therefore a low likelihood that said patient will either be asymptomatic or respond well to therapy.

15. The system of claim 14, wherein said machine readable instructions are further adapted to quantify and normalize at least one Phosphatidylcholine with Acyl-Alkyl Residue.

16. The system of claim 15, wherein said machine readable instructions are further adapted to quantify and normalize a plurality of Phosphatidylcholine with Acyl-Alkyl Residue.

17. The system of claim 14, wherein said first ratio comprises said Tyrosine to said Phenylalanine.

18. The system of claim 16, wherein said plurality of Phosphatidylcholine with Acyl-Alkyl Residue are selected from group of Arachdonic Phosphatidylcholine with Acyl-Alkyl Residue.

19. The system of claim 18, wherein said equation further comprises a summation of said plurality of Phosphatidylcholine with Acyl-Alkyl Residue.

20. The system of claim 19, wherein said equation further comprises a second ratio, said first ratio comprising Tyrosine to said Phenylalanine and said second ratio comprises said first ratio to said summation of said plurality of Phosphatidylcholine with Acyl-Alkyl Residue.
